(19) United States
(12) Patent Application Publication
(10) Pub. No.: US 2019/0125052 A1
(43) Pub. Date: May 2, 2019

(54) METHOD FOR TREATING HAIR

(71) Applicant: KAO CORPORATION, Tokyo (JP)

(72) Inventors: Jun-ichi FURUKAWA, Tokyo (JP); Shinichi TOKUNAGA, Tokyo (JP); Steven BREAKSPEAR, Darmstadt (DE); Niu JIAN, Darmstadt (DE); Bernd NOECKER, Darmstadt (DE)

(73) Assignee: KAO CORPORATION, Tokyo (JP)

(21) Appl. No.: 16/306,532

(22) PCT Filed: Jun. 2, 2017
(86) PCT No.: PCT/EP2017/063484

§ 371 Date: Nov. 30, 2018

(30) Foreign Application Priority Data
Jun. 3, 2016 (JP) 2016-112273

(51) Int. Cl. A45D 7/06; A61Q 5/06; A61Q 5/02; A61K 8/34; A61K 8/365; A61K 8/49

(52) U.S. Cl. CPC A45D 7/06; A61Q 5/06; A61Q 5/065; A45D 2007/001; A61K 8/347; A61K 8/365; A61K 8/498; A61Q 5/02

(57) ABSTRACT

The hair treatment method includes the following steps (i) to (iii) for dyeing hair and at the same time deforming hair semi-permanently or permanently:
(i) a step of dyeing hair through a hair treatment including applying a dye to the hair,
(ii) a step of applying the following components (A), (B), and (C) to the hair,
   (A): one or more compounds selected from the group consisting of glyoxylic acid, a glyoxylic acid hydrate, a glyoxylate salt, and a glyoxylamide;
   (B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);
   (C): water, and
(iii) a step of heating and shaping the hair.

### FIELD OF THE INVENTION

The present invention relates to a method for treating hair (hereinafter may be also referred to as a “hair treatment method”), which method can shape hair semi-permanently or permanently and also dye the hair in a single treatment process.

### BACKGROUND OF THE INVENTION

In semi-permanent or permanent deforming of hair, the hair is treated with a hair treatment composition containing a hair-reducing agent or with a strongly alkaline treatment agent. In dyeing hair to any of various colors, the hair is treated with a hair coloring (or dyeing) agent containing an oxidative dye or a direct dye. Generally, in the case where both hair deforming and dyeing are carried out, the hair is treated with a permanent agent or the like and then with a hair dyeing agent. Such an operation requires a very long time, which reduces the turnover of clients of a beauty salon. In addition, the operation imposes a load on a client who receives the hair treatment and results in double cost. Also, when hair receives separately performed a treatment with a permanent agent or the like and a subsequent treatment with a hair coloring agent, the hair may be severely damaged. Thus, conventional hair treatment processes have the aforementioned problems.

Under such circumstances, various techniques have been adopted to mitigate the hair damage by such a chemical treatment in deforming and dyeing hair. Patent Document 1 discloses a method for dyeing and permanently deforming hair, the method including sequentially applying an aqueous oxidative hair dye composition containing an oxidative dye precursor, an aqueous composition containing a reducing agent, a composition containing an oxidizing agent, and an aqueous oxidative hair dye composition containing an oxidative dye precursor, to thereby sequentially treat keratin fiber. Patent Document 2 discloses a method for simultaneously dyeing and permanently deforming hair, the method including mixing an oxidative hair dye composition containing a specific coupler and a specific developer with a permanent composition containing a keratin-reducing agent, and immediately after mixing, treating hair with the obtained mixture. Patent Document 3 discloses a two-step method for dyeing and semi-permanently straightening hair, the method sequentially including dyeing of hair by use of a first composition containing a hair dye, an optional alkaline agent, and an optional oxidizing agent (i.e., a dye composition) and, immediately after the dyeing treatment, straightening of the hair by use of a second composition containing a specific carboxylic acid (i.e., a straightening composition). Patent Document 4 discloses a two-step method for semi-permanently straightening and dyeing hair, the method sequentially including straightening of hair by use of a first composition containing a specific carboxylic acid (i.e., a straightening composition) and, immediately after thereof, dyeing of the hair by use of a second composition containing a hair dye, an optional alkaline agent, and an optional oxidizing agent (i.e., a dye composition).
Patent Document 1: EP-A-2020255
Patent Document 2: EP-A-1287812
Patent Document 3: WO2014/068102
Patent Document 4: WO2014/067702

### SUMMARY OF THE INVENTION

The present invention provides a hair treatment method which includes the following steps (i) to (iii) for dyeing hair and for deforming hair semi-permanently or permanently:

(i) a step of dyeing hair through a hair treatment including applying a dye to the hair,
(ii) a step of applying the following components (A), (B), and (C) to the hair,

(A): one or more compounds selected from the group consisting of glyoxylic acid, a glyoxylic acid hydrate, a glyoxylate salt, and a glyoxylamide;

(B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);

(C): water, and

(iii) a step of heating and shaping the hair.

### DETAILED DESCRIPTION OF THE INVENTION

The techniques disclosed in Patent Documents 1 and 2 require employment of a hair reducing agent that can break S—S bonds of protein in hair shafts and recombine the broken bonds. Thus, damage of hair is not satisfactory mitigated. In some cases, the hair dye is released from damaged portions of the hair, and color fastness to a daily hair caring procedure such as shampooing is unsatisfactorily. Also, in the method disclosed in Patent Document 2, the possible combination of the coupler and the developer of the oxidative dye is limited to specific ones, which is problematic. Meanwhile, the methods of Patent Documents 3 and 4 are satisfactory in terms of hair straightening effect. However, these techniques are not satisfied in the case where a client receives hair deforming to various desired shapes including curly hair and wavy hair.

In view of the foregoing, the present invention is directed to a hair treatment method, which gives less damage to hair; which can semi-permanently or permanently impart to hair a straight form, a wavy form, and a curly form, and simultaneously dye the deformed hair; which ensures excellent effect for deforming the shape of hair and color fastness against a treatment such as shampooing; and which can semi-permanently or permanently change the shaped hair shape to another hair shape in a simple way without using a hair treatment composition such as a reducing agent and giving no damage to the hair.

The present inventors have found that a hair deforming agent containing glyoxylic acid in combination with a specific phenolic compound can semi-permanently straighten hair or impart a curly shape of a wavy shape to the hair; and can change the hair shape to any shape different therefrom only by means of heating means such as a hair iron or a curler without performing a treatment by use of a hair treatment composition such as a reducing agent, when the hair has been treated with the hair cosmetic composition. The inventors have also found that, through a procedure sequentially including a first hair treatment with a hair dye composition and a second hair treatment with a hair deforming agent containing glyoxylic acid in combination with a specific phenolic compound, hair can be colored and semi-permanently or permanently deformed, while color fastness against a treatment such as shampooing is ensured, without impairing the effect for deforming the shape of hair and re-deforming effect intrinsic to the aforementioned hair deforming agent. The present invention has been accomplished on the basis of these findings.

As used herein, the expression “deforming of hair semi-permanently or permanently” refers to the shape of hair being maintained after repeated shampooing processes (i.e., with excellent shampooing resistance). More specifically, the expression and an equivalent expression refer to, when the deformed hair is washed with a shampoo, sufficiently rinsed off with water, and naturally dried, the shape of the hair is unchanged before and after shampooing. Notably, the expression “the shape of hair is not changed” refers to, for example, no substantial change in the number of waves of wavy hair before and after shampooing, or no substantial generation of wavy hair or curled hair after shampooing straight hair.

As used herein, the term “deforming of hair (or hair deforming)” refers to a change in shape of a hair shaft via no scission or recombination of S—S bonds of a hair-component protein, and includes a change from straight hair to curly hair or the like, and a change from wavy hair, curly hair, or the like as well as naturally frizzy hair or the like to straight hair.

Hair Dye Composition

Step (i) of the hair treatment method of the present invention is carried out by applying a hair dye composition to hair. The hair dye composition used in the present invention contains at least one dye, and optionally, at least one alkaline agent or an acid, and also optionally, at least one oxidizing agent. Examples of the mentioned at least one dye include at least one direct dye, at least one oxidative dye, and a combination thereof. This means that the hair dye composition may be a direct dye composition or an oxidative dye composition.

The hair dye composition may have any pH value, so long as it falls within a pH range normally employed for hair dye compositions. However, in order to make the effect of the present invention more prominent, the pH is preferably 6 or higher, more preferably 6.5 or higher, still more preferably 6.8 or higher. However, in order to attain excellent color lasting, an acidic hair dye composition is preferred.

### [Direct Dye]

No particular limitation is imposed on the type of the direct dye, and any direct dye suitable for hair coloring may be used. Examples of the direct dye include an anionic dye, a nitro dye, a disperse dye, a cationic dye, dyes which have azophenol structure selected from the group consisting of the following HC Red 18, HC Blue 18, and HC Yellow 16, a salt thereof, and a mixture of any of these.

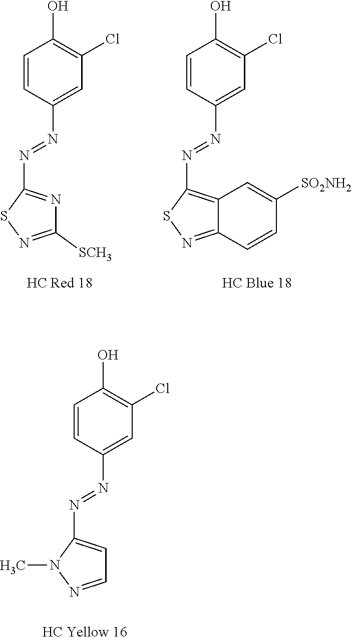 [SMILES: CSc1nsc(/N=N/c2ccc(O)c(Cl)c2)n1 ; NS(=O)(=O)c1ccc2nsc(/N=N/c3ccc(O)c(Cl)c3)c2c1 ; Cn1nccc1/N=N/c1ccc(O)c(Cl)c1]

Non-limitative examples of the cationic dye include Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green 1, Basic Orange 31, Basic Red 2, Basic Red 12, Basic Red 22, Basic Red 51, Basic Red 76, Basic Violet 1, Basic Violet 2, Basic Violet 3, Basic Violet 10, Basic Violet 14, Basic Yellow 57, Basic Yellow 87, and a mixture thereof. Of these, Basic Red 51, Basic Orange 31, Basic Yellow 87, and a mixture thereof are particularly preferred.

Non-limitative examples of the anionic dye include Acid Black 1, Acid Blue 1, Acid Blue 3, Food Blue 5, Acid Blue 7, Acid Blue 9, Acid Blue 74, Acid Orange 3, Acid Orange 4, Acid Orange 6, Acid Orange 7, Acid Orange 10, Acid Red 1, Acid Red 14, Acid Red 18, Acid Red 27, Acid Red 33, Acid Red 50, Acid Red 52, Acid Red 73, Acid Red 87, Acid Red 88, Acid Red 92, Acid Red 155, Acid Red 180, Acid Violet 2, Acid Violet 9, Acid Violet 43, Acid Violet 49, Acid Yellow 1, Acid Yellow 10, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet No. 2, D&C Yellow No. 7, D&C Yellow No. 8, D&C Yellow No. 10, FD&C Red No. 2, FD&C Red No. 40, FD&C Red No. 4, FD&C Yellow No. 6, FD&C Blue No. 1, Food Black 1, Food Black 2, an alkali metal (e.g., sodium or potassium) salt, and a mixture of any of these.

Of these, preferred anionic dyes are Acid Black 1, Acid Red 52, Acid Violet 2, Acid Violet 43, Acid Red 33, Acid Orange 4, Acid Orange 7, Acid Red 27, Acid Yellow 3, Acid Yellow 10, and a salt thereof. More preferred anionic dyes are Acid Red 52, Acid Violet 2, Acid Red 33, Acid Orange 4, Acid Yellow 10, a salt thereof, and a mixture of any of these.

Non-limitative examples of the nitro dye include HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HC Blue No. 12, HC Blue No. 13, HC Brown No. 1, HC Brown No. 2, HC Green No. 1, HC Orange No. 1, HC Orange No. 2, HC Orange No. 3, HC Orange No. 5, HC Red BN, HC Red No. 1, HC Red No. 3, HC Red No. 7, HC Red No. 8, HC Red No. 9, HC Red No. 10, HC Red No. 11, HC Red No. 13, HC Red No. 54, HC Red No. 14, HC Violet BS, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, 2-amino-6-chloro-4-nitrophenol, picramic acid, 1,2-diamino-4-nitrobenzole, 1,4-diamino-2-nitrobenzole, 3-nitro-4-aminophenol, 1-hydroxy-2-amino-3-nitrobenzole, 2-hydroxyethylpicramic acid, and a mixture thereof.

Non-limitative examples of the disperse dye include Disperse Blue 1, Disperse Black 9, Disperse Violet 1, and a mixture thereof.

These direct dyes may be used singly or in combination of two or more species. Thus, the hair dye composition employed in the present invention may contain therein a plurality of direct dyes having different ionic types.

From the viewpoint of attaining sufficient hair dyeing performance, the direct dye content of the hair dye composition used in the present invention, relative to the total composition of the hair dye composition, is preferably 0.001 mass % or higher, more preferably 0.01 mass % or higher, still more preferably 0.05 mass % or higher, and, from the viewpoint of blending of components, preferably 10 mass % or lower, more preferably 7.5 mass % or lower, still more preferably 5.0 mass % or lower, yet more preferably 3.0 mass % or lower.

In the case in which the hair dye composition contains only a direct dye as the dye, an oxidizing agent for dyeing the hair is not required. However, if a light hair color is desired, the hair dye composition may contain an oxidizing agent.

### [Oxidative Dye]

When the hair dye composition contains an oxidative dye, the composition is generally in the form of a two-agent type composition in which the first agent contains oxidative dye intermediates (a precursor and a coupler) and an alkaline agent, and the second agent contains an oxidizing agent such as hydrogen peroxide. Generally, the two agents are stored separately, and mixed together before application of the composition to hair.

No particular limitation is imposed on the type of the oxidative dye intermediates employed in the present invention, and any of known precursors and couplers generally employed in hair coloring products can be suitably used.

Non-limitative examples of the precursor include p-phenylenediamine, toluene-2,5-diamine, 2-chloro-p-phenylenediamine, N-methoxyethyl-p-phenylenediamine, N-phenyl-p-phenylenediamine, N,N-bis(2-hydroxyethyl)-p-phenylenediamine, 2-(2-hydroxyethyl)-p-phenylenediamine, 2,6-dimethyl-p-phenylenediamine, 4,4′-diaminodiphenylamine, 1,3-bis(N-(2-hydroxyethyl)-N-(4-aminophenyl)amino)-2-propanol, PEG-3,3,2′-p-phenylenediamine, p-aminophenol, p-methylaminophenol, 3-methyl-4-aminophenol, 2-aminomethyl-4-aminophenol, 2-(2-hydroxyethylaminomethyl)-4-aminophenol, o-aminophenol, 2-amino-5-methylphenol, 2-amino-6-methylphenol, 2-amino-5-acetamidophenol, 3,4-diaminobenzoic acid, 5-aminosalicylic acid, 2,4,5,6-tetraaminopyrimidine, 2,5,6-triamino-4-hydroxypyrimidine, 4,5-diamino-1-(4′-chlorobenzyl)pyrazole, 4,5-diamino-1-hydroxyethylpyrazole, a salt thereof, and a mixture of any of these.

Non-limitative examples of the coupler include m-phenylenediamine, 2,4-diaminophenoxyethanol, 2-amino-4-(2-hydroxyethylamino)anisole, 2,4-diamino-5-methylphenetole, 2,4-diamino-5-(2-hydroxyethoxy)toluene, 2,4-dimethoxy-1,3-diaminobenzene, 2,6-bis(2-hydroxyethylamino)toluene, 2,4-diamino-5-fluorotoluene, 1,3-bis(2,4-diaminophenoxy)propane, m-aminophenol, 2-methyl-5-aminophenol, 2-methyl-5-(2-hydroxyethylamino)phenol, 2,4-dichloro-3-aminophenol, 2-chloro-3-amino-6-methylphenol, 2-methyl-4-chloro-5-aminophenol, N-cyclopentyl-m-aminophenol, 2-methyl-4-methoxy-5-(2-hydroxyethylamino)phenol, 2-methyl-4-fluoro-5-aminophenol, p-amino-o-cresol, resorcin, 2-methylresorcin, 4-chlororesorcin, 1-naphthol, 1,5-dihydroxynaphthalene, 1,7-dihydroxynaphthalene, 2,7-dihydroxynaphthalene, 2-isopropyl-5-methylphenol, 4-hydroxyindole, 5-hydroxyindole, 6-hydroxyindole, 7-hydroxyindole, 6-hydroxybenzomorpholine, 3,4-methylenedioxyphenol, 2-bromo-4,5-methylenedioxyphenol, 3,4-methylenedioxyaniline, 1-(2-hydroxyethyl)amino-3,4-methylenedioxybenzene, 2,6-dihydroxy-3,4-dimethylpyridine, 2,6-dimethoxy-3,5-diaminopyridine, 2,3-diamino-6-methoxypyridine, 2-methylamino-3-amino-6-methoxypyridine, 2-amino-3-hydroxypyridine, 2,6-diaminopyridine, a salt thereof, and a mixture of any of these.

The precursor content or the coupler content of the hair dye composition employed in the present invention, relative to the total hair dye composition, is preferably 0.01 mass % or higher, more preferably 0.1 mass % or higher, and preferably 10 mass % or lower, more preferably 7.5 mass % or lower, still more preferably 5 mass % or lower.

The oxidative dye composition further contains an alkaline agent. Non-limitative examples of suitable alkaline agents include ammonia and a salt thereof; an alkanolamine (e.g., monoethanolamine, isopropanolamine, 2-amino-2-methylpropanol, or 2-aminobutanol) and a salt thereof; an alkanediamine (e.g., 1,3-propanediamine) and a salt thereof; a carbonate salt (e.g., guanidinium carbonate, sodium carbonate, potassium carbonate, sodium hydrogencarbonate, or potassium hydrogencarbonate); and a mixture thereof.

The alkaline agent content of the hair dye composition employed in the present invention, relative to the total hair dye composition, is preferably 0.1 mass % or higher, more preferably 0.5 mass % or higher, still more preferably 1 mass % or higher, and preferably 15 mass % or lower, more preferably 10 mass % or lower, still more preferably 7.5 mass % or lower.

The oxidizing agent-containing composition (second agent) of the oxidative dye composition and the dye-containing composition (first agent) are stored separately, and are mixed together before application to hair. Non-limitative examples of suitable oxidizing agents include hydrogen peroxide, urea peroxide, melamine peroxide, and sodium brominate. Among these oxidizing agents, hydrogen peroxide is preferred.

The oxidizing agent content of the hair dye composition employed in the present invention, relative to the total hair dye composition, is preferably 1 mass % or higher, more preferably 2 mass % or higher, and preferably 15 mass % or lower, more preferably 12 mass % or lower, still more preferably 9 mass % or lower.

In the case of separate storage, the pH of the oxidizing agent-containing composition (second agent) is preferably adjusted to 2 or higher, more preferably 2.5 or higher, and preferably 6 or lower, more preferably 4 or lower. The pH may be adjusted with a suitable buffer.

The pH (measured at room temperature (25° C.)) of the hair dye composition after mixing of the first agent and the second agent is preferably 6 or higher, more preferably 6.5 or higher, still more preferably 6.8 or higher, and preferably 11 or lower, more preferably 10.5 or lower, still more preferably 10 or lower.

The oxidative dye composition may further contain at least one direct dye. All of the aforementioned direct dyes are suited for the purpose.

The hair dye composition may suitably contain additional components such as the surfactants and/or conditioning components as defined below, whereby products of the composition in the form or solution, emulsion, cream, paste, or mousse may be provided.

### [Surfactant]

The hair dye composition may contain a surfactant. As the surfactant, any one of a cationic surfactant, a nonionic surfactant, an amphoteric surfactant, and an anionic surfactant may be used. Two or more types of surfactants may be used in combination.

The cationic surfactant is preferably a quaternary mono-long-chain alkylammonium salt having one C8 to C24 alkyl group and three C1 to C4 alkyl groups.

Preferably, at least one quaternary mono-long-chain alkylammonium surfactant is selected from the compounds represented by the following formula:

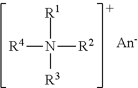

wherein R¹ represents a C8 to C22 saturated or unsaturated linear-chain or branched-chain alkyl group, R⁵—CO—NH—(CH₂)ₘ—, or R⁵—CO—O—(CH₂)ₘ— (wherein R⁵ represents a C7 to C21 saturated or unsaturated linear-chain or branched-chain alkyl chain, and m is an integer of 1 to 4); R², R³, and R⁴ each independently represent a C1 to C4 alkyl group or a C1 to C4 hydroxylalkyl group; and An⁻ represents a chloride ion, a bromide ion, a methosulfate ion, or an ethosulfate ion.

Examples of preferred cationic surfactants include long-chain quaternary ammonium compounds such as cetyltrimethylammonium chloride, myristyltrimethylammonium chloride, behenyltrimonium chloride, cetyltrimethylammonium bromide, steartrimonium chloride, and stearamidopropyltrimonium chloride. These compounds may be used singly or as a mixture thereof.

Examples of the nonionic surfactant include polyoxy-C₁₋₄-alkylene-C₈₋₂₄ alkyl ether, polyoxy-C₁₋₄-alkylene-C₈₋₂₄ alkenyl ether, higher (C₁₂-C₂₄) fatty acid sucrose ester, polyglycerin C₈₋₂₄ fatty acid ester, higher (C₁₂-C₂₄) fatty acid mono- or di-ethanolamide, polyoxyethylene hydrogenated castor oil, polyoxyethylene sorbitan C₈₋₂₄ fatty acid ester, polyoxyethylene sorbitan C₈₋₂₄ fatty acid ester, C₈₋₂₄ alkylsaccharide, C₈₋₂₄ alkylamine oxide, and C₈₋₂₄ alkylamidoamine oxide.

Examples of the amphoteric surfactant include imidazoline, carbobetaine, amidobetaine, sulfobetaine, hydroxysulfobetaine, and amidosulfobetaine.

Examples of the anionic surfactant include alkyl benzenesulfonate, alkyl or alkenyl ether sulfate, alkyl or alkenyl sulfate, olefin sulfonate, alkanesulfonate, saturated or unsaturated fatty acid salts, alkyl or alkenyl ether carboxylate, α-sulfofatty acid salts, N-acylamino acid, phosphoric acid mono- or di-ester, and sulfosuccinic acid salts. Examples of alkyl ether sulfate include polyoxyethylene alkyl ether sulfate.

Examples of counter ions of the anionic groups of these anionic surfactants include alkali metal ions (such as sodium ion and potassium ion); alkaline earth metal ions (such as calcium ion and magnesium ion); ammonium ion; and alkanolamine having one to three alkanol groups, each having 2 or 3 carbon atoms (for example, monoethanolamine, diethanolamine, triethanolamine, triisopropanolamine, and the like).

The surfactants may be used singly or in combination of two or more species. When a surfactant is incorporated into the hair dye composition employed in the present invention, the content of the surfactant is preferably 0.05 mass % or more, more preferably 0.1 mass % or more, and preferably 10 mass % or less, more preferably 5 mass % or less, relative to the total hair dye composition.

### [Conditioning Components]

The hair dye composition used in the present invention may optionally contain a conditioning component which is suitable for the hair. The conditioning component is an oil or a polymer which adheres to hair fiber to thereby improve feel to the touch and manageability of the hair.

When one or more conditioning components are employed, the total amount thereof is preferably 0.01 mass % or more, more preferably 0.05 mass % or more, yet more preferably 0.1 mass % or more, and preferably 30 mass % or less, more preferably 20 mass % or less, yet more preferably 10 mass % or less, relative to the total hair dye composition.

Examples of the conditioning component generally include silicones, higher alcohols, and organic conditioning oils (such as hydrocarbon oils, polyolefins, and fatty acid esters). The hair dye composition may contain a single species of a conditioning component, or a combination of two or more species.

### Silicone

The hair dye composition used in the present invention preferably contains silicone in order to improve the sensation during use. Examples of the silicone include dimethylpolysiloxane and modified silicones (such as amino-modified silicone, fluorine-modified silicone, alcohol-modified silicone, polyether-modified silicone, epoxy-modified silicone, and alkyl-modified silicone). Of these, preferred are dimethylpolysiloxane, polyether-modified silicone, and amino-modified silicone.

As the dimethylpolysiloxanes, any cyclic or noncyclic dimethylpolysiloxane polymer may be used. Examples thereof include SH200 series, BY22-019, BY22-020, BY11-026, B22-029, BY22-034, BY22-050A, BY22-055, BY22-060, BY22-083, and FZ-4188 (these are products of Dow Corning Toray), and KF-9008, KM-900 series, MK-15H, and MK-88 (these are products of Shin-etsu Chemical).

As the amino-modified silicones, any of silicones having an amino or ammonium group may be used. Examples include amino-modified silicone oils having terminal hydroxyl groups, some or all of which have been end-capped with, for example, a methyl group, and a non-end-capped amodimethicone. Preferred examples of the amino-modified silicones include those compounds represented by the following formula:

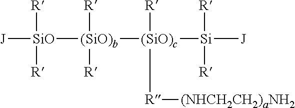

wherein R′ represents a hydrogen atom, a hydroxyl group, or R^{z}; R^{z} represents a substituted or non-substituted C1 to C20 monovalent hydrocarbon group; J represents R^{z}, R″—(NHCH₂CH₂)ₐNH₂, OR^{z}, or a hydroxyl group; R″ represents C1 to C8 divalent hydrocarbon group; a is a number of 0 to 3; and b+c (number average) is 10 or greater and less than 20,000, preferably 20 or greater and less than 3,000, more preferably 30 or greater and less than 1,000, still more preferably 40 or greater and less than 800.

Specific examples of preferred amino-modified silicone commercial products include amino-modified silicone oils such as SF8452C and SS3551 (products of Dow Corning Toray), KF-8004, KF-867S, and KF-8015 (products of Shin-Etsu Chemical Co., Ltd.), and amodimethicone emulsions such as SM8704C, SM8904, BY22-079, FZ-4671, and FZ-4672 (products of Dow Corning Toray).

The total silicone content of the hair dye composition, relative to the total composition of the hair dye composition, is preferably 0.1 mass % or higher, more preferably 0.2 mass % or higher, still more preferably 0.5 mass % or higher, and preferably 20 mass % or lower, more preferably 10 mass % or lower, still more preferably 5 mass % or lower.

### Oil Component

For improving the feel upon use, the hair dye composition of the present invention may contain an organic conditioning oil. The organic conditioning oil that is suitably used as a conditioning component is preferably a low-viscosity and water-insoluble liquid, and is selected from a hydrocarbon oil having at least 10 carbon atoms, a polyolefin, a fatty acid ester, a fatty acid amide, a polyalkylene glycol, and mixtures thereof. The viscosity of such an organic conditioning oil (as measured at 40° C.) is preferably 1 mPa*s or higher, more preferably 2 mPa*s or higher, and, preferably 200 mPa*s or lower, more preferably 100 mPa*s or lower, and even more preferably 50 mPa*s. For the determination of the viscosity, a capillary viscometer may be used.

Examples of the hydrocarbon oil include a cyclic hydrocarbon, a linear aliphatic hydrocarbon (saturated or unsaturated), and a branched aliphatic hydrocarbon (saturated or unsaturated), and polymers or mixtures thereof are also included. The linear hydrocarbon oil preferably has 12 to 19 carbon atoms. The branched hydrocarbon oil includes hydrocarbon polymers which preferably has more than 19 carbon atoms.

The polyolefin is a liquid polyolefin, more preferably a liquid poly-α-olefin, yet more preferably a hydrogenated liquid poly-α-olefin. The polyolefin used herein is prepared by polymerizing an olefin monomer having 4 to 14 carbon atoms, and preferably 6 to 12 carbon atoms.

The fatty acid ester may be, for example, a fatty acid ester having at least 10 carbon atoms. Examples of such a fatty acid ester include esters having a hydrocarbon chain derived from a fatty acid and an alcohol (for example, monoesters, polyhydric alcohol esters, or di- and tricarboxylic acid esters). The hydrocarbon group of these fatty acid esters may have another compatible functional group (such as an amide group or an alkoxy group) as a substituent, or the hydrocarbon group may be covalently bonded to those functional groups. More specifically, an alkyl and alkenyl ester of a fatty acid having a fatty acid chain having 10 to 22 carbon atoms, a carboxylic acid ester of an aliphatic alcohol having an aliphatic chain derived from an alkyl and/or alkenyl alcohol having 10 to 22 carbon atoms, and a mixture thereof are suitably used. Specific examples of these preferred fatty acid esters include isopropyl isostearate, hexyl laurate, isohexyl laurate, isohexyl palmitate, isopropyl palmitate, decyl oleate, isodecyl oleate, hexadecyl stearate, decyl stearate, dihexadecyl adipate, lauryl lactate, myristyl lactate, cetyl lactate, oleyl stearate, oleyl oleate, oleyl myristate, lauryl acetate, cetyl propionate and dioleyl adipate.

Further suitable oil components are natural oils such as paraffin oil and natural triglycerides.

Suitable natural triglycerides are argan oil, Shea butter oil, karite oil, olive oil, almond oil, avocado oil, ricinus oil, coconut oil, palm oil, sesame oil, peanut oil, sunflower oil, peach kernel oil, wheat germ oil, macadamia nut oil, macadamia oil, night primrose oil, jojoba oil, castor oil, soya oil, lanolin, passiflora oil, black cumin oil, borage oils, grapeseed oil, hempseed oil, kukui nut oil, and rosehip oil.

The organic conditioning oil may be used in combination of two or more kinds, and the total concentration is preferably 0.1 mass % or higher, more preferably 0.2 mass % or higher, yet more preferably 0.5 mass % or higher, based on the total composition of the hair dye composition, and preferably 20 mass % or lower, more preferably 10 mass % or lower, yet more preferably 5 mass % or lower.

### Alcohols

From the viewpoint of improving the feel to the touch and stability, the hair dye composition may also contain a higher alcohol having 8 or more carbon atoms. Typically, the higher alcohol has 8 to 22 carbon atoms, and preferably 16 to 22 carbon atoms. Specific examples thereof include cetyl alcohol, stearyl alcohol, behenyl alcohol, and mixtures thereof.

The higher alcohol may be used in combination of two or more kinds, and the content thereof is preferably 0.1 mass % or higher, more preferably 0.2 mass % or higher, yet more preferably 0.5 mass % or higher, based on the total composition of the hair dye composition, and, preferably 20 mass % or less, more preferably 10 mass % or less, even more preferably 5 mass % or less.

Polyols may also be incorporated suitably into the hair dye composition. Examples of the polyalkylene glycol include polyethylene glycol and polypropylene glycol, and a mixture of the two may be used, or a copolymer of ethylene oxide and propylene oxide may also be used.

Besides, the hair dye composition may contain further components conventionally used in the field of cosmetics, such as preservatives, chelating agents, stabilizers, oxidation inhibitors, plant extracts, ultraviolet absorbers, vitamins, dyes, and fragrances.

Hair Deforming Agent

Step (ii) of the hair treatment method of the present invention is carried out by applying a hair deforming agent to hair. The hair deforming agent used in the present invention encompasses a one-agent type hair deforming agent composed of a single agent, and a multi-agent type hair deforming agent composed of multiple agents (e.g., of a 2-agent type). The multi-agent type hair deforming agent is categorized into a single-application type, which is applied to hair after mixing a first agent, a second agent, and other components, and a successive-application type, whose first agent, second agent, and the like is successively applied to hair.

As used herein, the term “the total composition of hair deforming agent” refers to, in the case of a one-agent type hair deforming agent, the single agent forming the one-agent type hair deforming agent, and in the case of a single application multi-agent type hair deforming agent, a mixture containing all the compositions forming the multi-agent type hair deforming agent, at such compositional proportions that fall within the scope of the present invention, with the compositions being mixed before application of hair. In the case of a successive application multi-agent type hair deforming agent, which is not an actual mixture before application, “the total composition of hair deforming agent” refers to a conceptual mixture containing all the compositions forming the multi-agent type hair deforming agent, at such compositional proportions that fall within the scope of the present invention.

The hair deforming agent used in the present invention contains the following components (A) to (C) in its total composition:

(A): one or more compounds selected from the group consisting of glyoxylic acid, glyoxylic acid hydrate, glyoxylate salt, or a glyoxylamide,

(B): a phenolic compound having an electron-donating group on at least one m-position (preferably on two m-positions) and having a hydrogen atom on at least one of the o-positions and the p-position (wherein the electron-donating group on the m-position may form, together with an adjacent carbon atom, a benzene ring which may be further substituted with hydroxyl group(s)),

(C): water.

### [Component (A): Glyoxylic Acid, Glyoxylic Acid Hydrate, Glyoxylate Salt, or a Glyoxylamide]

Component (A) includes any of glyoxylic acid, a glyoxylic acid hydrate, a glyoxylate salt, and a glyoxylamide. Examples of the glyoxylic acid hydrate include glyoxylic acid monohydrate. Examples of the glyoxylate salt include an alkali metal glyoxylate and an alkaline earth metal glyoxylate. Examples of the alkali metal include lithium, sodium, and potassium, and examples of the alkaline earth metal include magnesium and calcium. Examples of the glyoxylamide include N-glyoxyloylcarbocysteine and N-glyoxyloylkeratinamino acid.

From the viewpoints of enhancing a effect for deforming the shape of hair of the hair deforming agent, further enhancing resistance of the hair shape against shampooing, further enhancing the re-deforming effect of the hair which has been semi-permanently deformed via heating, and further enhancing the resistance to shampooing after re-deforming of the hair, the hair deforming agent preferably has a component (A) content, relative to the total composition of the hair deforming agent and in terms of glyoxylic acid, of 1.0 mass % or higher, more preferably 2.0 mass % or higher, still more preferably 2.5 mass % or higher, yet more preferably 3.0 mass % or higher. In addition to the aforementioned viewpoints, from a further viewpoint of suppressing irritation to the skin, the component (A) content is preferably 30 mass % or less, more preferably 25 mass % or less, still more preferably 20 mass % or less, yet more preferably 15 mass % or less, further more preferably 12 mass % or less.

### [Component (B): Phenolic Compound Having a Specific Structure]

Component (B) is a phenolic compound having an electron-donating group on at least one m-position (preferably on two m-positions) and having a hydrogen atom on at least one of the o-positions and the p-position. The electron-donating group on the m-position may form, together with an adjacent carbon atom, a benzene ring which may be further substituted with hydroxyl group(s). From the viewpoint of permeability to hair, the molecular weight of component (B) is preferably 100 or higher, more preferably 110 or higher, and 1,000 or lower, more preferably 700 or lower, still more preferably 500 or lower. Examples of the phenolic compound of component (B) include the following components (B1), (B2), and (B3):

(B1) resorcin;
(B2) a compound represented by formula (1); and
(B3) a compound represented by formula (2).

Component (B1) is a resorcin, represented by the following formula.

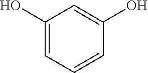

Component (B2) is a compound represented by formula (1):

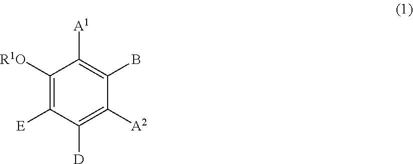

wherein:

R¹ represents a hydrogen atom or a methyl group;

A¹ and A², which may be identical to or different from each other, each represent a hydrogen atom, a C1 to C12 linear-chain or branched-chain alkyl or alkenyl group, an optionally substituted C7 to C12 aralkyl or arylalkenyl group, a C1 to C6 linear-chain or branched-chain alkoxy or alkenyloxy group, a halogen atom, or —CO—R² (wherein R² represents a C1 to C12 linear-chain or branched-chain alkyl or alkenyl group, an optionally substituted C7 to C12 aralkyl or arylalkenyl group, or an optionally substituted C6 to C12 aromatic hydrocarbon group);

B represents a hydrogen atom, a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group);

D represents a hydrogen atom, a hydroxyl group, a methyl group, or a C1 to C12 linear-chain or branched-chain alkoxy or alkenyloxy group; and

E represents a hydrogen atom, a hydroxyl group, a C1 to C6 linear-chain or branched-chain alkyl or alkenyl group, or a C1 to C6 linear-chain or branched-chain alkoxy or alkenyloxy group,

wherein two or three of A¹, A², B, and E each are a hydrogen atom, with each of remaining being a group which is not a sulfo group, and in the case where D is a hydrogen atom or a methyl group, A¹ and B, or A² and B form an optionally hydroxyl group-substituted benzene ring with two adjacent carbon atoms.

In the case where the aralkyl group, the arylalkenyl group, or the aromatic hydrocarbon group in represented by formula (1) have a substituent, examples of the substituent include a hydroxyl group, a C1 to C6 linear-chain or branched-chain alkyl or alkenyl group, and a C1 to C12 alkoxy group. The number of carbon atoms (Cn) of the aralkyl group, the arylalkenyl group, or the aromatic hydrocarbon group refers to the total number of the carbon atoms including those forming the substituents.

Examples of the C1 to C6 linear-chain or branched-chain alkyl or alkenyl group of R³ or E include methyl, ethyl, n-propyl, isopropyl, n-butyl, isobutyl, sec-butyl, tert-butyl, n-pentyl, isopentyl, neopentyl, 1-methylpentyl, n-hexyl, isohexyl, vinyl, allyl, butenyl, and hexenyl.

Examples of the C1 to C6 linear-chain or branched-chain alkoxy or alkenyloxy group of A¹, A², or E include groups in which the aforementioned C1 to C6 alkyl or alkenyl group to which an oxygen atom is bonded.

Examples of the C1 to C12 linear-chain or branched-chain alkyl or alkenyl group in A¹, A², R², and B include the aforementioned C1 to C6 alkyl or alkenyl groups, n-heptyl, 2,4-dimethylpentyl, 1-n-propylbutyl, n-octyl, 2-ethylhexyl, n-nonyl, 1-methylnonyl, n-decyl, 3,7-dimethyloctyl, 2-isopropyl-5-methylhexyl, n-undecyl, n-dodecyl, and decenyl.

Examples of the C1 to C12 linear-chain or branched-chain alkoxy or alkenyloxy group of D include groups formed of the aforementioned C1 to C12 alkyl or alkenyl group to which an oxygen atom is bound.

Examples of the optionally substituted C7 to C12 aralkyl or arylalkenyl group of A¹, A², R², and B include benzyl, hydroxybenzyl, dihydroxybenzyl, phenylethyl, phenylethenyl, hydroxyphenylethyl, dihydroxyphenylethyl, hydroxyphenylethenyl, dihydroxyphenylethenyl, phenylpropyl, phenylpropenyl, phenylbutyl, phenylbutenyl, phenylpentyl, phenylpentenyl, phenylhexyl, and phenylhexenyl.

Examples of the optionally substituted C6 to C12 aromatic hydrocarbon group of R² include phenyl, hydroxyphenyl, dihydroxyphenyl, trihydroxyphenyl, naphthyl, hydroxynaphthyl, and dihydroxynaphthyl.

Examples of the halogen atom of A¹ and A² include fluorine, chlorine, and bromine.

Specific examples of the compound represented by formula (1) include a resorcin derivative represented by formula (1-1), a benzophenone derivative represented by formula (1-2), and a naphthol derivative represented by formula (1-3-a) or (1-3-b), which are shown below.

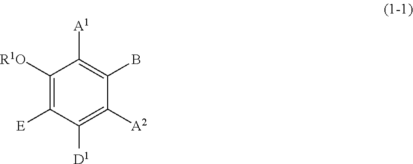

wherein R¹, A¹, A², B, and E are defined as above, and D¹ represents a hydroxyl group or a methoxy group

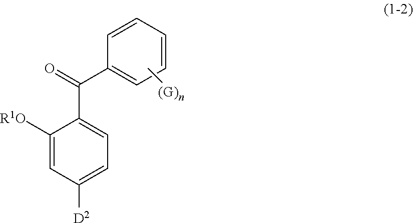

wherein R¹ has the same meaning as defined above; D² represents a hydroxyl group or a C1 to C12 alkoxy group; G represents a hydroxyl group, a C1 to C6 linear-chain or branched-chain alkyl or alkenyl group, or a C1 to C6 alkoxy group; and n is an integer of 0 to 2

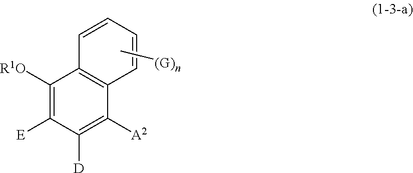

wherein R¹, A², E, D, G, and n are defined as above.

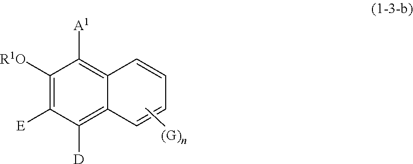

wherein R¹, A¹, E, D, G, and n are defined as above.

The compound represented by formula (1-1) is preferably any of the compounds (1-1-1) to (1-1-3).

(1-1-1) m-Dimethoxybenzene Derivative Represented by the Following Formula (1-1-1):

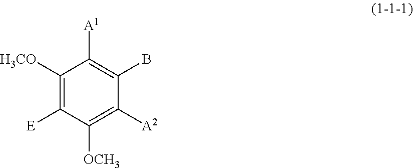

wherein A¹, A², B, and E are defined as above.

Each of A¹ and A² is preferably a hydrogen atom, a C1 to C4 linear-chain or a branched-chain alkyl or alkenyl group, more preferably a hydrogen atom.

B is preferably a hydrogen atom, a C1 to C4 alkyl or alkenyl group, an optionally substituted C7 to C10 arylalkenyl group, or a hydroxyl group, more preferably a hydrogen atom, an optionally substituted C7 to C10 arylalkenyl group, or a hydroxyl group.

E is preferably a hydrogen atom, a C1 to C4 linear-chain or branched-chain alkyl or alkenyl group, more preferably a hydrogen atom.

Examples of the compound (1-1-1) include 1,3-dimethoxybenzene, 3,5-dimethoxyphenol, 2,6-dimethoxyphenol, and 5-(hydroxyphenylethenyl)-1,3-dimethoxybenzene (trivial name: pterostilbene).

(1-1-2) m-Methoxyphenol Derivative Represented by the Following Formula (1-1-2):

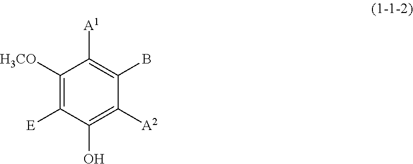

wherein A¹, A², B, and E are defined as above.

Each of A¹ and A² is preferably a hydrogen atom, a C1 to C12 linear-chain or branched-chain alkyl or alkenyl group, or an optionally substituted C7 to C12 aralkyl or arylalkenyl group, more preferably a hydrogen atom, a C1 to C6 linear-chain or branched-chain alkyl group, or an optionally substituted C7 to C10 arylalkenyl group.

B is preferably a hydrogen atom, a C1 to C12 linear-chain or branched-chain alkyl or alkenyl group, an optionally substituted C7 to C12 aralkyl or arylalkenyl group, or —OR³ (wherein R³ is a hydrogen atom or a C1 to C6 linear-chain or branched-chain alkyl or alkenyl group), more preferably a hydrogen atom, a C1 to C4 alkyl or alkenyl group, an optionally substituted C7 to C10 arylalkenyl group, or a hydroxyl group, still more preferably a hydrogen atom, an optionally substituted C7 to C10 arylalkenyl group, or a hydroxyl group.

E is preferably a hydrogen atom, a hydroxyl group, a C1 to C4 linear-chain or branched-chain alkyl or alkenyl group, or a C1 to C4 linear-chain or branched-chain alkoxy or alkenyloxy group, more preferably a hydrogen atom or a hydroxyl group.

Examples of the compound (1-1-2) include 3-methoxyphenol, 5-methoxyresorcin, 3-methoxybenzene-1,2-diol, 4-butyl-3-methoxyphenol, 3-methoxy-4-(1-phenylethyl)phenol, and 5-(4-hydroxyphenylethenyl)-1-hydroxy-3-methoxybenzene (trivial name: pinostilbene).

### (1-1-3) Resorcin Derivative Represented by the Following Formula (1-1-3):

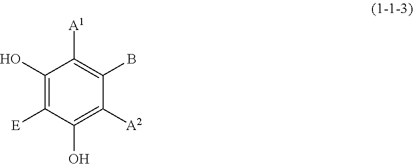

wherein A¹, A², B, and E are defined as above.

Examples of the resorcin derivative represented by formula (1-1-3) include the resorcin derivatives represented by the following formula (i) or (ii).

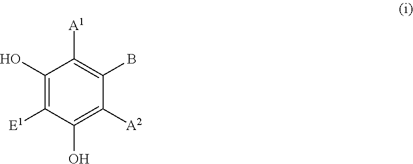

wherein A¹, A², and B are defined as above; E¹ represents a hydroxyl group, a C1 to C6 linear-chain or branched-chain alkyl or alkenyl group, or a C1 to C6 linear-chain or branched-chain alkoxy or alkenyloxy group

Each of A¹ and A² is preferably a hydrogen atom or a C1 to C12 linear-chain or branched-chain alkyl or alkenyl group, more preferably a hydrogen atom.

B is preferably a hydrogen atom, an optionally substituted C7 to C12 aralkyl or arylalkenyl group, or —OR³ (wherein R³ is a hydrogen atom or a C1 to C4 linear-chain or branched-chain alkyl or alkenyl group).

E¹ is preferably a hydroxyl group, a C1 to C4 linear-chain or branched-chain alkyl or alkenyl group, or a C1 to C4 linear-chain or branched-chain alkoxy or alkenyloxy group.

Examples of the resorcin derivative represented by formula (i) include 2-alkylresorcins such as 2-methylresorcin, 2-ethylresorcin, and 2-propylresorcin; pyrogallol; 2-alkoxyresorcins such as 2-methoxyresorcin; gallate esters such as gallic acid, methyl gallate, ethyl gallate, propyl gallate, and butyl gallate; and 5-(phenylethenyl)-2-isopropylresorcin.

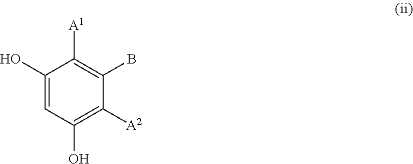

wherein A¹, A², and B are defined as above.

The resorcin derivative represented by formula (ii) is more preferably resorcin derivatives represented by formula (ii-1) or (ii-2).

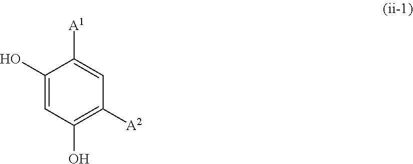

wherein A¹ and A² are defined as above.

Examples of the resorcin derivative represented by formula (ii-1) include

4-alkylresorcins such as 4-methylresorcin, 4-ethylresorcin, 4-propylresorcin, 4-isopropylresorcin, 4-butylresorcin (trivial name: rucinol), 4-isobutylresorcin, 4-sec-butylresorcin, 4-tert-butylresorcin, 4-pentylresorcin, 4-isopentylresorcin, 4-sec-pentylresorcin, 4-tert-pentylresorcin, 4-neopentylresorcin, 4-hexylresorcin, 4-isohexylresorcin, 4-heptylresorcin, 4-octylresorcin, 4-(2-ethylhexyl)resorcin, 4-nonylresorcin, 4-decylresorcin, 4-undecylresorcin, and 4-dodecylresorcin;

4-alkenylresorcins such as 4-vinylresorcin, 4-allylresorcin, 4-butenylresorcin, 4-hexenylresorcin, and 4-decenylresorcin;

4-aralkylresorcins such as 4-benzylresorcin, 4-(1-phenylethyl)resorcin (trivial name: Symwhite 377), 4-furanylethylresorcin, 4-tetrahydropyranylresorcin, 4-(2-phenylethyl)resorcin, and 4-(3-phenylpropyl)resorcin;

4-hydroxyaralkylresorcins such as 4-(4-hydroxybenzyl)resorcin, 4-(2,4-dihydroxybenzyl)resorcin, 4-(4-hydroxyphenylethyl)resorcin, and 4-(2,4-dihydroxyphenylethyl)resorcin;

4-arylalkenylresorcins such as 4-(1-phenylethenyl)resorcin and 4-(3-phenylpropenyl)resorcin;

4-hydroxyarylalkenylresorcins such as 4-(4-hydroxyphenylethenyl) resorcin and 4-(2,4-dihydroxyphenylethenyl)resorcin;

4-(1-methylnaphthyl)resorcin;

4-alkoxyresorcins such as 4-methoxyresorcin, 4-ethoxyresorcin, 4-isopropoxyresorcin, 4-propoxyresorcin, 4-butoxyresorcin, 4-sec-butoxyresorcin, 4-tert-butoxyresorcin, and 4-pentoxyresorcin;

haloresorcins such as 4-chlororesorcin and 4-bromoresorcin;

4-alkanoylresorcins such as 4-acetylresorcin, 4-propanoylresorcin, 4-butanoylresorcin, 4-pentanoylresorcin, and 4-hexanoylresorcin; and

4-arylalkanoylresorcins such as 4-phenylethanoylresorcin, 4-phenylpropanoylresorcin, 4-phenylbutanoylresorcin, 4-phenylpentanoylresorcin, 4-phenylhexanoylresorcin, 3-(hydroxyphenyl)-1-(2,4-dihydroxyphenyl)propen-1-one (trivial name: isoliquiritigenin).

Among these, from the viewpoint of obtaining more remarkable change of the shape of hair after the treatment using the hair cosmetic composition of the present invention, improving shampooing resistance of shaped hair, obtaining more remarkable change of the shape of hair during semipermanent re-deforming of the shape of hair by heating, and improving shampooing resistance of shaped hair after re-deforming of the shape of hair by means of condensate of component (A) and component (B) formed in the hair, one or m ore member selected from the group consisting of 4-alkylresorcin, 4-aralkylresorcin and 4-halogenated resorcin is preferable, and one or more member selected from the group consisting of 4-hexyl resorcin, Rucinol, Symwhite and 4-chlororesorcin is more preferable.

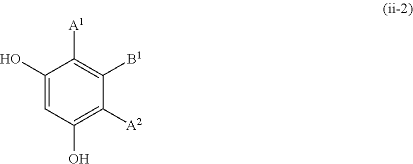

wherein A¹ and A² are defined as above; B² represents a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group).

The resorcin derivative represented by formula (ii-2) is more preferably resorcin derivatives represented by formula (ii-2-a) or (ii-2-b).

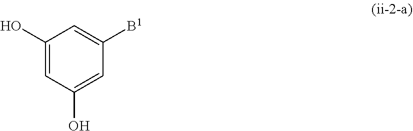

wherein B¹ has the same meaning as defined above.

Examples of the resorcin derivative represented by formula (ii-2-a) include

5-alkylresorcins such as 5-methylresorcin, 5-ethylresorcin, 5-propylresorcin, 5-isopropylresorcin, 5-butylresorcin, 5-isobutylresorcin, 5-sec-butylresorcin, 5-tert-butylresorcin, 5-pentylresorcin (trivial name: olivetol), 5-isopentylresorcin, 5-neopentylresorcin, 5-hexylresorcin, 5-isohexylresorcin, 5-heptylresorcin, 5-octylresorcin, 5-(2-ethylhexyl)resorcin, 5-nonylresorcin, 5-decylresorcin, 5-undecylresorcin, and 5-dodecylresorcin;

5-alkenylresorcins such as 5-vinylresorcin, 5-allylresorcin, 5-butenylresorcin, 5-hexenylresorcin, and 5-decenylresorcin;

phloroglucinol;

5-alkoxybenzene-1,3-diols such as 5-ethoxybenzene-1,3-diol, 5-propoxybenzene-1,3-diol, and 5-butoxybenzene-1,3-diol;

3,5-dihydroxybenzoic acid;

3,5-dihydroxybenzoate esters such as methyl 3,5-dihydroxybenzoate, ethyl 3,5-dihydroxybenzoate, propyl 3,5-dihydroxybenzoate, butyl 3,5-dihydroxybenzoate, pentyl 3,5-dihydroxybenzoate, and hexyl 3,5-dihydroxybenzoate;

5-aralkylresorcins such as 5-benzylresorcin, 5-(1-phenylethyl)resorcin, 5-(2-phenylethyl)resorcin, and 5-(phenylpropyl) resorcin;

5-hydroxyaralkylresorcins such as 5-(4-hydroxybenzyl)resorcin, 5-(2,4-dihydroxybenzyl)resorcin, 5-(hydroxyphenylethyl)resorcin (trivial name: dihydroresveratrol), and 5-(2,4-dihydroxyphenylethyl)resorcin;

5-arylalkenylresorcins such as 5-(phenylethenyl)resorcin (trivial name: pinosylvin) and 5-(phenylpropenyl)resorcin; and

5-hydroxyarylalkenylresorcins such as 5-(4-hydroxyphenylethenyl)resorcin (trivial name: resveratrol), 5-(4-methoxyphenylethenyl)resorcin (trivial name: 4-methoxyresveratrol), 5-(2,4-dihydroxyphenylethenyl)resorcin (trivial name: oxyresveratrol), 5-(2-methoxy-4-hydroxyphenylethenyl)resorcin (trivial name: gnetucleistol D), 5-(3,4-dimethoxyphenylethenyl)resorcin (trivial name: gnetucleistol E), 5-(3-hydroxy-4-methoxyphenylethenyl)resorcin (trivial name: rhapontigenin), 5-(4-hydroxy-3-methoxyphenylethenyl)resorcin (trivial name: isorhapontigenin), and 5-(dihydroxyphenylethenyl)resorcin (trivial name: piceatannol).

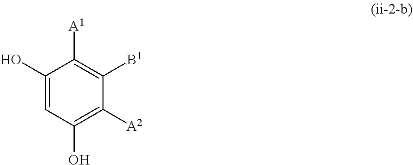

wherein A¹, A², and B¹ are defined as above.

Each of A¹ and A² is preferably a hydrogen atom, a C1 to C4 linear-chain or branched-chain alkyl or alkenyl group, or a C1 to C4 alkoxy or alkenyloxy group.

Examples of the resorcin derivative represented by formula (ii-2-b) include

2-alkylbenzene-1,3,5-triols such as 2-methylbenzene-1,3,5-triol, 2-ethylbenzene-1,3,5-triol, 2-propylbenzene-1,3,5-triol, 2-butylbenzene-1,3,5-triol, 2-hexylbenzene-1,3,5-triol, 2-octylbenzene-1,3,5-triol, and 2-dodecylbenzene-1,3,5-triol;

2-aralkyl-1,3,5-triols such as 2-benzylbenzene-1,3,5-triol, 2-(phenylethyl)benzene-1,3,5-triol, and 2-(phenylpropyl)benzene-1,3,5-triol;

2,4,6-trihydroxyphenylaralkylketone such as 2-acetylbenzene-1,3,5-triol, 2-propanoylbenzene-1,3,5-triol, 2-butanoylbenzene-1,3,5-triol, 2-phenylethanoylbenzene-1,3,5-triol, 2-hydroxyphenyl-1-(benzene-2,4,6-triol)ethan-1-one, 3-hydroxyphenyl-1-(benzene-2,4,6-triol)propan-1-one (trivial name: phloretin), 4-hydroxyphenyl-1-(benzene-2,4,6-triol)butan-1-one, 2-benzoylbenzene-1,3,5-triol, 2-(hydroxybenzoyl)benzene-1,3,5-triol, 2-(3,5-dihydroxybenzoyl)benzene-1,3,5-triol, and 2-(2,4-dihydroxybenzoyl)benzene-1,3,5-triol; and

3,5-dihydroxybenzoate esters such as 3,5-dihydroxy-2-methylbenzoic acid, methyl 3,5-dihydroxy-2-methylbenzoate, 3,5-dihydroxy-2-ethylbenzoic acid, methyl 3,5-dihydroxy-2-ethylbenzoate, 3,5-dihydroxy-2-propylbenzoic acid, methyl 3,5-dihydroxy-2-propylbenzoate, 3,5-dihydroxy-2-butylbenzoic acid, and methyl 3,5-dihydroxy-2-butylbenzoate.

Examples of the benzophenone derivative represented by formula (1-2) include 4-benzoylresorcin (trivial name: Benzophenone-1), 4-(hydroxybenzoyl)resorcin, 4-(dihydroxybenzoyl)resorcin, 4-(2,4-dihydroxybenzoyl)resorcin (trivial name: Benzophenone-2), 4-(methylbenzoyl)resorcin, 4-(ethylbenzoyl)resorcin, 4-(dimethylbenzoyl)resorcin, 4-(diethylbenzoyl) resorcin, 4-naphthoylresorcin, 2-hydroxy-4-methoxybenzophenone (trivial name: Benzophenone-3), 2,2′-dihydroxy-4, 4′-dimethoxybenzophenone (trivial name: Benzophenone-6), 2,2′-dihydroxy-4-methoxybenzophenone (trivial name: Benzophenone-8), 2-hydroxy-4-methoxy-4′-methylbenzophenone (trivial name: Benzophenone-10), and 2-hydroxy-4-octyloxybenzophenone (trivial name: Benzophenone-12).

Among the naphthol derivatives represented by formula (1-3-a) or (1-3-b), preferred are those represented by formula (1-3-a) or (1-3-b) in which R¹ is a hydrogen atom or a C1 to C4 alkyl or alkenyl group, with a hydrogen atom being more preferred.

Also, in the above naphthol derivatives, each of A¹ and A² is preferably a hydrogen atom, a hydroxyl group, a C1 to C4 linear-chain or branched-chain alkyl group, or a C1 to C4 alkoxy group, more preferably a hydrogen atom or a hydroxyl group.

Also, in the above naphthol derivatives, D is preferably a hydrogen atom, a hydroxyl group, a C1 to C4 linear-chain or branched-chain alkyl group, or a C1 to C4 alkoxy group.

Also, in the above naphthol derivatives, E is preferably a hydrogen atom, a hydroxyl group, a C1 to C4 alkyl group, or a C1 to C4 alkoxy group.

Examples of the above compounds include 1-naphthol, 2-naphthol, 3-methylnaphthalen-1-ol, naphthalene-1,4-diol, naphthalene-1,5-diol, and naphthalene-1,8-diol.

Among the compounds represented by formula (1), preferred are m-dimethoxybenzene derivatives represented by formula (1-1-1), resorcin derivatives represented by formula (1-1-3), benzophenone derivatives represented by formula (1-2), and naphthol derivative represented by formula (1-3-a) or (1-3-b). More preferred compounds are 2-methylresorcin, 4-chlororesorcin, 4-alkylresorcin, 4-aralkylresorcin, 4-acylated resorcin, 5-alkylresorcin, 5-aralkylresorcin, 5-hydroxyarylalkenylresorcin, 2,4,6-trihydroxyphenylaralkylketone, gallic acid, and a gallate ester. Still more preferred compounds are 4-butylresorcin (trivial name: rucinol), 4-hexylresorcin, 4-(1-phenylethyl)resorcin (trivial name: Symwhite 377), 4-furanylethylresorcin, 4-tetrahydropyranylresorcin, 5-(hydroxyphenylethenyl)resorcin (trivial name: resveratrol), 3-hydroxyphenyl-1-(benzene-2,4,6-triol)propan-1-one (trivial name: phloretin), 4-(2,4-dihydroxybenzoyl)resorcin (trivial name: Benzophenone-2), 5-(hydroxyphenylethenyl)-1,3-dimethoxybenzene (trivial name: pterostilbene), and 1-naphthol. Yet more preferred compounds are 2-methylresorcin, 4-chlororesorcin, 1-naphthol, 4-n-butylresorcin, 4-(1-phenylethyl)resorcin, 5-(hydroxyphenylethenyl) resorcin, 3-hydroxyphenyl-1-(benzene-2,4,6-triol)propan-1-one, and 4-(2,4-dihydroxybenzoyl)resorcin.

In addition, among the compound represented by general formula (1), obtaining more remarkable change of the shape of hair of ter the treatment using the hair cosmetic composition of the present invention, as well as improving restoration of damaged hair damaged by chemical treatment, by means of condensate of component (A) and component (B) formed in the hair, one or more member selected from the group consisting of m-dimethoxybenzene derivatives represented by general formula (1-1-1), resorcin derivatives represented by general formula (1-1-3), benzophenone derivatives represented by general formula (1-2), and naphthol derivatives represented by general formula (1-3-a) or (1-3-b) is preferable.

Furthermore, one or more member selected from the group consisting of 2-alkylresorcin, 4-alkylresorcin, 4-aralkyl resorcin, 4-halogenated resorcin, 5-hydroxyarylalkenylresorcin, 2,4,6-trihydroxyphenylaralkylketone, benzophenone derivative, naphthol, 4-acylated resorcin, 5-alkylresorcin, 5-aralkylresorcin, gallic acid and gallic acid ester is preferable.

Furthermore, one or more member selected from the group consisting of 2-methylresorcin, 4-butylresorcin (trivial name: Rucinol), 4-hexylresorcin, 4-(1-phenylethyl)resorcin (trivial name: Symwhite377), 4-chlororesorcin, 5-(hydroxyphenylethenyl)resorcin (trivial name: resveratrol), 5-(hydroxyphenylethenyl)-1,3-dimeth oxybenzene (trivial name: Pterostilbene), 3-hydroxyphenyl-1-(benzene-2,4,6-triol)propane-1-on (trivial name: Phloretin), 4-(2,4-dihydroxybenzoyl)resorcin (trivial name: Benzophenone-2) and 1-naphthol is preferable.

The compound represented by formula (1) preferably has a molecular weight of 120 or higher, and from the viewpoint of permeability to hair, 1,000 or lower, more preferably 500 or lower, still more preferably 300 or lower.

Component (B3) is a compound represented by the following formula (2).

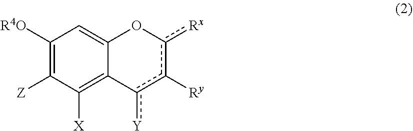

wherein,

R⁴ represents a hydrogen atom or a methyl group;

X represents a hydrogen atom, a hydroxyl group, or a methoxy group;

Y represents a hydrogen atom, an oxygen atom, a hydroxyl group, or a methoxy group;

Z represents a hydrogen atom or a C1 to C5 linear-chain or branched-chain alkyl or alkenyl group;

R^{x} represents a hydrogen atom, an oxygen atom, a hydroxyl group, a methoxy group, or an aromatic hydrocarbon group which may be substituted with 1 to 3 hydroxyl groups or methoxy groups and which optionally forms a condensed ring with 1,3-dioxolane;

R^{y} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups;

a dotted line portion may be a double bond;

each of the combinations of the dotted line and the solid line being adjacent to R^{x} or Y denotes a double bond in the case where R^{x} or Y is an oxygen atom, and denotes a single bond in the other cases; and

Z represents a C1 to C5 linear-chain or branched-chain alkyl or alkenyl group, only when R^{x} or R^{y} is an o,p-dihydroxyaromatic hydrocarbon group, and represents a hydrogen atom in the other cases

Examples of the C1 to C5 linear-chain or branched-chain alkyl or alkenyl group of Z include methyl, ethyl, n-propyl, isopropyl, n-butyl, isobutyl, sec-butyl, tert-butyl, n-pentyl, isopentyl, sec-pentyl, tert-pentyl, neopentyl, 1-methylpentyl, vinyl, allyl, and butenyl.

Examples of the aromatic hydrocarbon group of R^{x} or R^{y} include phenyl and naphthyl. Examples of the aromatic hydrocarbon group which forms a condensed ring with 1,3-dioxolane include 1,3-benzodioxol-5-yl group.

Examples of the arylcarbonyloxy group of R^{y} include a benzoyloxy group, and examples of the aralkylcarbonyloxy group of R^{y} include a benzylcarbonyloxy group, a phenylethylcarbonyloxy group, a phenylpropylcarbonyloxy group, and a phenylbutylcarbonyloxy group.

Specific examples of the compound represented by formula (2) include compounds represented by the following formulas (2-1) to (2-5).

### (2-1) Flavanols Represented by the Following Formula (2-1):

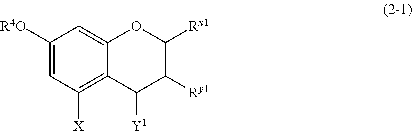

wherein

R⁴ and X are defined as above;

Y¹ represents a hydrogen atom, a hydroxyl group, or a methoxy group;

R^{x1} represents an aromatic hydrocarbon group which may be substituted with 1 to 3 hydroxyl groups or methoxy groups and which optionally forms a condensed ring with 1,3-dioxolane; and

R^{y1} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups.

### (2-2) Flavanones or Flavanonol Represented by the Following Formula (2-2):

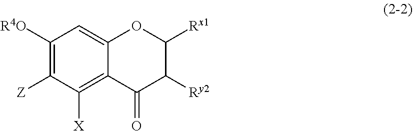

wherein R⁴, X, Z, and R^{x1} are defined as above, and R^{y2} represents a hydrogen atom, a hydroxyl group, or a methoxy group; and

Z represents a C1 to C5 linear-chain or branched-chain alkyl or alkenyl group, only when R^{x1} is an o,p-dihydroxyaromatic hydrocarbon group, and represents a hydrogen atom in the other cases

### (2-3) Flavanones or Flavanonol Represented by the Following Formula (2-3):

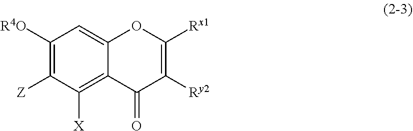

wherein R⁴, X, Z, R^{x1}, and R^{y2} are defined as above. Z is a C1 to C5 linear-chain or branched-chain alkyl or alkenyl group, only when R^{x1} is an o,p-dihydroxyaromatic hydrocarbon group. In the other cases, Z is a hydrogen atom.]

### (2-4) Isoflavons and Isoflavans Represented by the Following Formula (2-4):

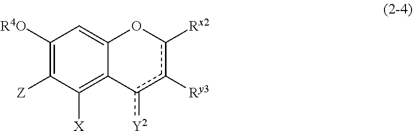

wherein

R⁴, X, Z, and the dotted line portion are defined as above;

Y² represents a hydrogen atom or an oxygen atom;

R^{x2} represents a hydrogen atom, a hydroxyl group, or a methoxy group;

R^{y3} represents an aromatic hydrocarbon group which may be substituted with 1 to 3 hydroxyl groups or methoxy groups and which optionally forms a condensed ring with 1,3-dioxolane;

the combination of the dotted line and the solid line being adjacent to Y² denotes a double bond in the case where Y² is an oxygen atom, and denotes a single bond in the other cases; and

Z represents a C1 to C5 linear-chain or branched-chain alkyl or alkenyl group, only when R^{y3} is an o,p-dihydroxyaromatic hydrocarbon group, and represents a hydrogen atom in the other cases

### (2-5) Coumarins Represented by Formula (2-5):

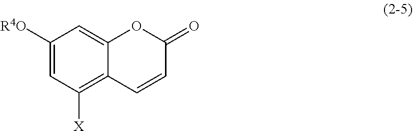

wherein R⁴ and X are defined as above.

The compound (2-1) is preferably compound represented by the following formula (2-1-A) to (2-1-C).

### (2-1-A) Flavan-3-Ols Represented by the Following Formula (2-1-A):

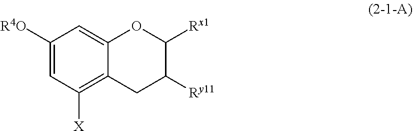

wherein R⁴, X, and R^{x1} are defined as above; R^{y11} represents a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups.

The flavan-3-ols represented by formula (2-1-A) are preferably those in which R⁴ and X are the same as defined above; R^{x1} is an aromatic hydrocarbon group which may be substituted with 1 to 3 hydroxyl groups or methoxy groups; R^{y1} is a hydrogen atom, a hydroxyl group, a methoxy group, or an arylcarbonyloxy or aralkylcarbonyloxy group which may be substituted with 1 to 3 hydroxyl groups or methoxy groups.

Examples of the compound of (2-1-A) include catechin, epicatechin, epigallocatechin, meciadanol, afzelechin, epiafzelechin, catechin gallate, epicatechin gallate, epigallocatechin gallate, phylloflavan, fisetinidol, guibourtinidol, and robinetinidol.

### (2-1-B) Flavan-4-Ols Represented by the Following Formula (2-1-B):

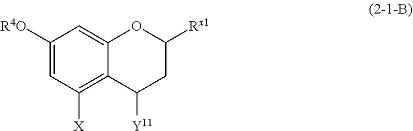

wherein R⁴, X, and R^{x1} are defined as above, and Y¹¹ represents a hydroxyl group or a methoxy group

The flavan-4-ol represented by formula (2-1-B) is preferably a compound wherein R⁴ and X are defined as above, Y¹¹ is a hydroxyl group, and R^{x1} is an aromatic hydrocarbon group which may be substituted with 1 to 3 hydroxyl groups or methoxy groups.

Examples of the compound of (2-1-B) include apiforol and luteoforol.

### (2-1-C) Flavan-3,4-Diols Represented by the Following Formula (2-1-C):

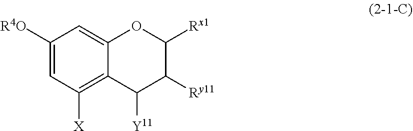

wherein R⁴, X, Y¹¹, R^{x1}, and R^{y11} are defined as above.

The flavan-3,4-diol represented by formula (2-1-C) is preferably a compound wherein R⁴ and X are defined as above; Y¹¹ is a hydroxyl group or a methoxy group; R^{x1} is an aromatic hydrocarbon group which may be substituted with 1 to 3 hydroxyl groups or methoxy groups; and R^{y1} is a hydroxyl group or a methoxy group.

Examples of the compound of (2-1-C) include leucocyanidin, leucodelphinidin, leucopelargonidin, leucopeonidin, and leucofisetinidin.

The compound of (2-2) is preferably any of the compounds represented by the following formula (2-2-A) or (2-2-B).

### (2-2-A) Flavanones Represented by the Following Formula (2-2-A):

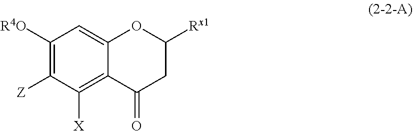

wherein R⁴, X, Z, and R^{x1} are defined as above, and

Z represents a C1 to C5 linear-chain or branched-chain alkyl or alkenyl group, only when R^{x1} is an o,p-dihydroxyaromatic hydrocarbon group, and represents a hydrogen atom in the other cases

The flavanone represented by formula (2-2-A) is preferably a compound wherein R⁴ and X are defined as above; Z is a hydrogen atom; and R^{x1} is an aromatic hydrocarbon group which may be substituted with 1 to 3 hydroxyl groups or methoxy groups; and R^{y1} is a hydroxyl group or a methoxy group.

Examples of the compound of (2-2-A) include eriodictyol, naringenin, pinocembrin, hesperetin, homoeriodictyol, isosakuranetin, sterubin, sakuranetin, alpinetin, and butin.

### (2-2-B) Flavanonols Represented by the Following Formula (2-2-B):

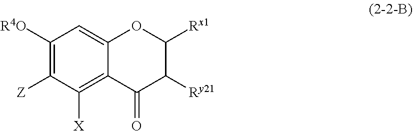

wherein R⁴, X, Z, and R^{x1} are defined as above; R^{y21} represents a hydroxyl group or a methoxy group;

Z represents a C1 to C5 linear-chain or branched-chain alkyl or alkenyl group, only when R^{x1} is an o,p-dihydroxyaromatic hydrocarbon group, and represents a hydrogen atom in the other cases

The flavanonol represented by formula (2-2-B) is preferably a compound wherein R⁴ and X are defined as above; Z is a hydrogen atom; R^{x1} is an aromatic hydrocarbon group which may be substituted with 1 to 3 hydroxyl groups or methoxy groups; and R^{y1} is a hydroxyl group or a methoxy group.

Examples of the compound of (2-2-B) include aromadendrin, taxifolin, and dihydrokaempferide.

The compound of (2-3) is preferably any of the compounds represented by the following formula (2-3-A) or (2-3-B).

### (2-3-A) Flavones Represented by the Following Formula (2-3-A):

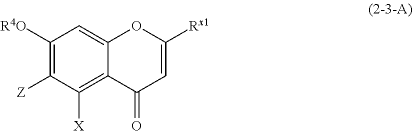

wherein R⁴, X, Z, and R^{x1} are defined as above; and

Z represents a C1 to C5 linear-chain or branched-chain alkyl or alkenyl group, only when R^{x4} is an o,p-dihydroxyaromatic hydrocarbon group, and represents a hydrogen atom in the other cases

The flavone represented by formula (2-3-A) is preferably a compound wherein R⁴ and X are defined as above; Z is a hydrogen atom; and R^{x1} is an aromatic hydrocarbon group which may be substituted with 1 to 3 hydroxyl groups or methoxy groups.

Examples of the compound of (2-3-A) include luteolin, apigenin, chrysin, norartocarpetin, tricetin, diosmetin, acacetin, chrysoeriol, genkwanin, techtochrysin, tricin, 4′,7-dihydroxyflavone, and pratol.

### (2-3-B) Flavonols Represented by Following Formula (2-3-B)

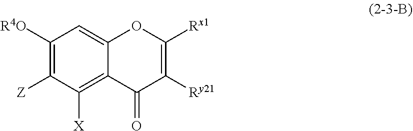

wherein R⁴, X, Z, R^{x1}, and R^{y21} are defined as above; and

Z represents a C1 to C5 linear-chain or branched-chain alkyl or alkenyl group, only when R^{x1} is an o,p-dihydroxyaromatic hydrocarbon group, and represents a hydrogen atom in the other cases

The flavonol represented by formula (2-3-B) is preferably a compound wherein R⁴ and X are defined as above; Z is a hydrogen atom; R^{x1} is an aromatic hydrocarbon group which may be substituted with 1 to 3 hydroxyl groups or methoxy groups; and R^{y1} is a hydroxyl group or a methoxy group.

Examples of the compound of (2-3-B) include quercetin, myricetin, morin, kaempferol, galangin, kaempferide, tamarixetin, laricitrin, annulatin, isorhamnetin, syringetin, rhamnetin, europetin, azaleatin, 5-O-methylmyricetin, retusin, pachypodol, rhamnazin, ayanin, ombuin, and fisetin.

The compound of (2-4) is preferably any of the compounds represented by the following formula (2-4-A) to (2-4-C).

### (2-4-A) Isoflavones Represented by the Following Formula (2-4-A):

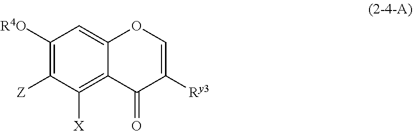

wherein R⁴, X, Z, and R^{y3} are defined as above; and

Z represents a C1 to C5 linear-chain or branched-chain alkyl or alkenyl group, only when R^{y3} is an o,p-dihydroxyaromatic hydrocarbon group, and represents a hydrogen atom in the other cases

The isoflavone represented by formula (2-4-A) is preferably a compound wherein R⁴ and X are defined as above; Z is a hydrogen atom or a C1 to C5 linear-chain or branched-chain alkyl or alkenyl group; and R^{y3} is an aromatic hydrocarbon group which may be substituted with 1 to 3 hydroxyl groups or methoxy groups and optionally forms a condensed ring with 1,3-dioxolane.

Examples of the compound of (2-4-A) include genistein, daidzein, orobol, biochanin A, pratensein, 5-O-methylgenistein, prunetin, calycosin, formononetin, 7-O-methylluteone, luteone, and pseudobaptigenin.

### (2-4-B) Isoflavans Represented by the Following Formula (2-4-B):

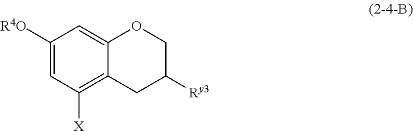

wherein R⁴, X, and R^{y3} are defined as above.

The isoflavan represented by formula (2-4-B) is preferably a compound wherein R⁴ and X are defined as above; and R^{y3} is an aromatic hydrocarbon group which may be substituted with 1 to 3 hydroxyl groups or methoxy groups and optionally forms a condensed ring with 1,3-dioxolane.

Examples of the compound of (2-4-B) include equol.

### (2-4-C) Isoflavenes Represented by the Following Formula (2-4-C):

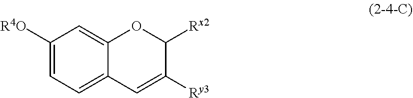

wherein R⁴, R^{x2}, and R^{y3} are defined as above.

The isoflavene represented by formula (2-4-C) is preferably a compound wherein R⁴ and R^{x2} are defined as above; and R^{y3} is an aromatic hydrocarbon group which may be substituted with 1 to 3 hydroxyl groups or methoxy groups and optionally forms a condensed ring with 1,3-dioxolane.

Examples of the compound of (2-4-C) include haginin D, haginin E, and 2-methoxyjudaicin.

Examples of the compound (2-5) include umbelliferone.

Among the aforementioned compounds represented by formula (2), preferred compounds are flavan-3-ols represented by formula (2-1-A), flavonols represented by formula (2-3-B), flavanones represented by formula (2-2-A), flavones represented by formula (2-3-A), isoflavones represented by formula (2-4-A), isoflavans represented by formula (2-4-B), and coumarins represented by formula (2-5). Specific examples of more preferred compounds include catechin, epicatechin, epigallocatechin, catechin gallate, epicatechin gallate, epigallocatechin gallate, quercetin, morin, hesperetin, naringenin, chrysin, daidzein, equol, and umbelliferone. Of these, catechin, epigallocatechin, epigallocatechin gallate, naringenin, and equol are yet more preferred. Mixtures such as a green tea, containing the aforementioned compounds, may also be used.

Each of the compounds represented by formula (2) preferably has a molecular weight of 150 or higher. From the viewpoint of permeability to hair, the molecular weight is preferably 1,000 or lower, more preferably 700 or lower, still more preferably 500 or lower.

Among the component (B), from the viewpoint of obtaining more remarkable change of the shape of hair after the treatment using the hair cosmetic composition of the present invention, improving shampooing resistance of shaped hair, obtaining more remarkable change of the shape of hair during semipermanent re-deforming of the shape of hair by heating, and improving shampooing resistance of shaped hair after re-deforming of the shape of hair by means of condensate of component (A) and component (B) formed in the hair, one or more member selected from the group consisting of resorcin derivatives represented by general formula (ii-1), m-dimethoxybenzene derivatives represented by general formula (1-1-1), resorcin derivatives represented by general formula (1-1-3), benzophenone derivatives represented by general formula (1-2), naphthol derivatives represented by general formula (1-3-a) or (1-3-b), flavan-3-ols represented by general formula (2-1-A), flavonols represented by general formula (2-3-B), flavanones represented by general formula (2-2-A), flavones represented by general formula (2-3-A), isoflavones represented by general formula (2-4-A), isoflavans represented by general formula (2-4-B) and coumarins represented by general formula (2-5) is preferable, and favorable compounds classified in each component is as described above.

Subcomponents of component (B) may be used singly or in combination of two or more species. Two or more of (B1) to (B3) may be used in combination. In the present invention, (B2) or (B3) is preferred, from the viewpoint of more consistently deforming hair.

In order to attain a remarkable change in hair shape after treatment with the hair deforming agent used in the present invention, to further enhance the resistance of the hair shape to shampooing, to attain a more remarkable change in hair shape upon semi-permanently re-deforming of hair through heating, and to further enhance the resistance of the re-deformed hair to shampooing, the hair deforming agent preferably has a total component (B) content, relative to the total hair deforming agent, of 0.2 mass % or higher, more preferably 0.5 mass % or higher, still more preferably 1.0 mass % or higher, yet more preferably 1.5 mass % or higher. In addition to the aforementioned viewpoints, in order to suitably mix components, the total component (B) content is preferably 40 mass % or less, more preferably 30 mass % or less, still more preferably 25 mass % or less, yet more preferably 23 mass % or less, further more preferably 20 mass % or less.

In the case where component (B1) is used as component (B), the component (B1) content (i.e., the preferred component (B) content) of the hair deforming agent, relative to the total hair deforming agent, is more preferably 2 mass % or higher, still more preferably 3 mass % or higher, yet more preferably 4 mass % or higher, further more preferably 5 mass % or higher, and in order to suitably mix components, more preferably 17 mass % or less.

In the case where component (B2) is used as component (B), the component (B2) content (i.e., the preferred component (B) content) of the hair deforming agent is more preferably 2 mass % or higher, and in order to suitably mix components, more preferably 17 mass % or less, still more preferably 15 mass % or less, yet more preferably 12 mass % or less.

In the case where component (B3) is used as component (B), the component (B3) content (i.e., the preferred component (B) content) of the hair deforming agent is more preferably 17 mass % or less, still more preferably 15 mass % or less, yet more preferably 12 mass % or less, in order to suitably mix components.

In order to attain a remarkable change in hair shape after treatment with the hair deforming agent by a condensation product between component (A) and component (B) formed in hair shafts, to further enhance the resistance of the hair shape to shampooing, to attain a more remarkable change in hair shape upon semi-permanently re-deforming of hair through heating, and to further enhance the resistance of the re-deformed hair to shampooing, the molar ratio of component (B) to component (A), (B)/(A), of the hair deforming agent applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.1 or lower, yet more preferably 1.9 or lower, further more preferably 1.7 or lower, further more preferably 1.6 or lower.

In the case where component (B1) is used as component (B), the molar ratio of component (B1) to component (A), (B1)/(A), of the hair deforming agent applied to hair is preferably 0.2 or higher, more preferably 0.3 or higher, still more preferably 0.4 or higher, yet more preferably 0.5 or higher, further more preferably 0.7 or higher, and preferably lower than 2.5, more preferably 2.3 or lower, still more preferably 2.0 or lower, yet more preferably 1.5 or lower, further more preferably 1.2 or lower.

In the case where component (B2) is used as component (B), the molar ratio of component (B2) to component (A), (B2)/(A), of the hair deforming agent applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2.0 or lower, yet more preferably 1.5 or lower, further more preferably 1.2 or lower.

In the case where component (B3) is used as component (B), the molar ratio of component (B3) to component (A), (B3)/(A), of the hair deforming agent applied to hair is preferably 0.001 or higher, more preferably 0.1 or higher, still more preferably 0.2 or higher, yet more preferably 0.25 or higher, and preferably lower than 2.5, more preferably 2.3 or lower, still more preferably 2 or lower, yet more preferably 1.5 or lower.

### [Component (C): Water]

The medium of the hair deforming agent is (C) water. In the case of the multi-agent type type, each of the first agent and the second agent employs (C) water as the medium.

The hair deforming agent may be of a one-agent type or a multi-agent type (e.g., a two-agent type) type. However, in order to enhance permeability of components (A) and (B) to hair to attain enhanced effects of the present invention, a multi-agent type composition in which component (A) and component (B) are contained in different agents and which is used in the format of successive application is preferred. Further, a two-agent type composition is more preferred. In a preferred mode of the successive application-type multi-agent type composition, components (B) and (C) are incorporated into the first agent, which is firstly applied to the hair, and components (A) and (C) are incorporated into the second agent, which is applied to the hair after application of the first agent.

[pH]

When the hair deforming agent is of a one-agent type, the pH of the agent is preferably 4 or lower, more preferably 3 or lower, still more preferably 2.5 or lower, yet more preferably 2 or lower, from the viewpoint of permeability to hair. Also, from the viewpoints of suppression of damage to the hair and irritation to the skin, the pH is preferably 1 or higher, more preferably 1.2 or higher, still more preferably 1.5 or higher. In the case of a multi-agent type, the pH of the agent containing component (A); i.e., the second agent, is preferably adjusted to fall within the aforementioned ranges. In the case of a multi-agent type composition, the pH of the agent containing component (B); i.e., the first agent, is preferably 6 or lower, more preferably 5 or lower, still more preferably 4.5 or lower, and preferably 2.5 or higher, more preferably 3 or higher, still more preferably 3.5 or higher, from the viewpoint of preventing discoloration of the composition. Notably, according to the present invention, the pH of the hair deforming agent refers to a pH value of a non-diluted or non-treated sample of the hair deforming agent measured at room temperature (25° C.) by means of a pH meter (model F-52, product of HORIBA).

In order to adjust the pH of the hair deforming agent to fall within the above ranges, a pH-adjusting agent may be appropriately used. Examples of the alkali pH-adjusting agent which may be used in the invention include ammonia and salts thereof; alkanolamines such as monoethanolamine, isopropanolamine, 2-amino-2-methylpropanol, and 2-aminobutanol, and salts thereof; alkane diamines such as 1,3-prpoanediamine, and salts thereof; carbonate salts such as guanidinium carbonate, sodium carbonate, potassium carbonate, sodium hydrogencarbonate, and potassium hydrogencarbonate; and hydroxides such as sodium hydroxide and potassium hydroxide. Examples of the acid pH-adjusting agent which may be used in the invention include inorganic acids such as hydrochloric acid and phosphoric acid; hydrochloric acid salts such as monoethanolamine hydrochloride; phosphate salts such as dihydrogen potassium phosphate and hydrogen disodium phosphate; organic acids other than component (A), such as lactic acid and malic acid.

### [Other Components]

In order to improve touch feeling to treated hair and to further enhance the effects of the present invention, one or more compositions forming the hair deforming agent may contain silicone and a cationic surfactant described as a component capable of being incorporated into a hair dye composition. The silicone amount in the hair deforming agent is preferably the amount that is described to be contained in the hair dye composition. When the hair deforming agent is of the multi-agent type, cationic surfactants and silicone may be contained in the first agent and/or the second agent.

In order to improve hair touch feeling after the hair treatment, one or more component compositions forming the hair deforming agent preferably contain a cationic polymer.

In the present invention, the “cationic polymer” refers to a polymer having a cationic group or a group which can be ionized to form a cationic group, and an amphoteric polymer having a totally positive charge. Examples of the cationic polymer include an aqueous polymer having an amino group or an ammonium group in polymer side chains and an aqueous polymer formed of structural repeating units of diallyl quaternary ammonium salt. Specific examples include cationized cellulose derivatives, cationic starch, cationic guar gum derivatives, diallyl quaternary ammonium salt polymers and copolymers, and quaternarized polyvinylpyrrolidone derivatives. Among them, from the viewpoints of attaining effects (mild touch feeling during rinsing and shampooing, smoothness and easy finger passage, high hair manageability during drying, and moisturizing effect) and stability of the treatment agents, polymers formed of structural repeating units of diallyl quaternary ammonium salt, quaternarized polyvinylpyrrolidone derivatives, and cationized cellulose derivatives are preferred, with diallyl quaternary ammonium salt polymers and copolymers and cationized cellulose derivatives being more preferred.

Specific examples of preferred diallyl quaternary ammonium salt polymers and copolymers include dimethyldiallylammonium chloride polymer (Polyquaternium-6) (e.g., Merquat 100; product of Lubrizol Advanced Materials), dimethyldiallylammonium chloride-acrylic acid copolymer (Polyquaternium-22) (e.g., Merquat 280 or 295; products of Lubrizol Advanced Materials), and dimethyldiallylammonium chloride-acrylamide copolymer (Polyquaternium-7) (e.g., Merquat 550; product of Lubrizol Advanced Materials).

Specific examples of preferred quaternarized polyvinylpyrrolidone derivatives include a polymer produced through polymerization of a vinylpyrrolidone copolymer with dimethylaminoethyl methacrylate (Polyquaternium-11) (e.g., Gafquat 734, Gafquat 755, or Gafquat 755N (products of Ashland).

Specific examples of preferred cationized cellulose include a polymer formed through addition of glycidyltrimethylammonium chloride to hydroxycellulose (Polyquaternium-10) (e.g., Leogard G or GP (products of Lion Specialty Chemicals Co., Ltd.), or Polymer JR-125, JR-400, JR-30M, LR-400, or LR-30M (products of Amerchol), and hydroxyethylcellulose dimethyldiallylammonium chloride (Polyquaternium-4) (e.g., Cell coat H-100 or L-200 (products of Akzo Nobel).

The cationic polymer content of the hair deforming agent, relative to the total composition of the hair deforming agent, is preferably 0.001 mass % or higher, more preferably 0.01 mass % or higher, still more preferably 0.05 mass % or higher, and preferably 20 mass % or lower, more preferably 10 mass % or lower. In the case where the hair deforming agent is of the multi-agent type, the cationic polymer may be contained in the first agent and/or the second agent.

Optionally, an antioxidant may be added to one or more components of the hair deforming agent. The type and amount of the antioxidant may be those generally employed in the field of hair deforming agents. An example thereof is ascorbic acid.

Preferably, the hair deforming agent contains substantially no hair reducing agent. A characteristic feature of the present invention is to realize hair deforming not through breakage of S—S bonds of a protein in hair shafts. Therefore, the present invention absolutely differs from a permanent wave agent which breaks S—S bonds of hair shafts by the action of a reducing agent. Examples of the hair reducing agent include thiols such as thioglycolic acid, dithioglycolic acid, cysteine, acetylcysteine, and butyrolactone thiol, hydrogen sulfite, and a salt thereof.

In the present specification, the expression “substantially containing no specific compound” or an equivalent expression refers to the hair deforming agent having a target compound content, relative to the total composition of the hair deforming agent, of preferably less than 0.1 mass %, more preferably less than 0.01 mass %. Yet more preferably, the hair deforming agent contains no target compound.

The hair deforming agent is highly safe to the human body and gives less damage to hair. Thus, the composition can be suitably applied particularly to human hair.

### [Hair Deforming Treatment Method]

According to the present invention, through carrying out a hair treatment method including the following steps (i) to (iii), hair can be dyed and at the same time deformed semi-permanently or permanently:

(i) a step of dyeing hair through a hair treatment including applying a dye to the hair,
(ii) a step of applying the following components (A), (B), and (C) to the hair,

(A): one or more compounds selected from the group consisting of glyoxylic acid, a glyoxylic acid hydrate, a glyoxylate salt, and a glyoxylamide;

(B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);

(C): water, and

(iii) a step of heating and shaping the hair.

### Step (i): Dyeing Step

Dyeing step (i) is carried out before step (ii), by applying a hair dye composition containing one or more dyes selected from the group consisting of direct dyes and oxidative dyes.

When the hair dye composition is a direct dye composition, the composition per se is applied to the hair, whereas when the hair dye composition is composed of a first agent containing an oxidative dye and a second agent containing an oxidizing agent, the two agents are mixed and the mixture is immediately applied to the hair. The temperature at which the dyeing composition is applied is 15 to 45° C. After the application to the hair, the composition is left on the hair for 1 to 60 minutes, preferably 5 to 45 minutes, more preferably 5 to 30 minutes, followed by washing out with water. Optionally, the hair may be shampooed and dried.

The ratio by mass of hair to the hair dyeing composition applied to the hair is preferably 0.5:2 to 2:0.5, more preferably 0.5:1 to 1:0.5, even more preferably approximately 1:1.

### Step (ii): Application Steps of Components (A) to (C)

In step (ii), components (A), (B), and (C) are applied to hair. Specifically, a hair deforming agent containing components (A), (B), and (C) is used according to the procedure described hereinbelow (note that in the following description of hair treatment method, when the expression “hair deforming agent” is used, it means a composition actually applied to hair, and encompasses any of a one-agent type hair deforming agent, a single application-type multi-agent type hair deforming agent which is a mixture of a first agent and a second agent, and a successive application-type multi-agent type hair deforming agent employing a first agent and a second agent.

Step (ii) may be performed after step (i) but before the effect of the hair dyeing treatment is lost. For example, step (ii) may be performed within a month after completion of the step (i) treatment. In order to retain the color of the dyed hair vividly, the time span between step (i) and step (ii) is preferably short, i.e., within 2 weeks, more preferably within 1 week, further more preferably within a few days, yet more preferably within one day, yet more preferably within 3 hours, yet more preferably within one hour, yet more preferably within 30 minutes, yet more preferably within 10 minutes, after completion of the step (i) treatment.

In the case of a one-agent type hair deforming agent:

(ii) a step of applying a one-agent type hair deforming agent to hair.

In the case of a multi-agent type hair deforming agent in a single application format:

(ii) a step of mixing a first agent and a second agent of a multi-agent type hair deforming agent and applying the obtained hair deforming agent to hair.

In the case of a multi-agent type hair deforming agent in a successive application format:

(ii) a step of applying a first agent of a multi-agent type hair deforming agent to hair and then applying a second agent of the hair deforming agent to a portion of the hair to which the first agent has been applied.

In step (ii), the hair deforming agent may be applied to dry hair or wet hair. However, the target hair is preferably wetted with water before step (ii) so as to swell the hair and promote permeation of the hair deforming agent to the hair. The relative mass of the hair deforming agent applied to hair in step (ii); i.e., the mass ratio of liquid to hair (mass of hair deforming agent/mass of hair) is preferably 0.05 or higher, more preferably 0.1 or higher, still more preferably 0.25 or higher, yet more preferably 0.5 or higher, and preferably 5 or lower, more preferably 3 or lower, still more preferably 2 or lower. In the case of the successive application-type hair deforming agent, from the viewpoint of applicability, each of the first agent and the second agent preferably satisfies the above liquid-to-hair conditions. The target hair may be the total hair of the head or a part of the hair.

In step (ii), when the successive application-type multi-agent type hair deforming agent is used, the first agent is firstly applied, and, in order to promote permeation of the hair deforming agent and enhance the effects of the invention, the hair to which the first agent of the hair deforming agent has been applied may be allowed to stand for a certain period of time, and then the second agent may be further applied thereto. For suitably causing the hair deforming agent to permeate and diffuse in hair, the time of allowing to stand is preferably 1 minute or longer, more preferably 3 minutes or longer, still more preferably 5 minutes or longer, and preferably 1 hour or shorter, more preferably 30 minutes or shorter, still more preferably 20 minutes or shorter. In this case, in order to promote permeation of the first agent, the hair may be heated. The heating temperature is preferably 40 to 90° C.

In step (ii), when the successive application-type multi-agent type hair deforming agent is used, a step of washing off the first agent (hereinafter may be referred to as an “intermediate rinsing step”) may be performed after application and staying of the first agent and before application of the second agent. From the viewpoint of shortening the total treatment time, the treatment method preferably includes no intermediate rinsing step. When no intermediate rinsing step is included, the molecular weight of component (B) contained in the first agent is preferably 100 to 180, more preferably 100 to 140, for further enhancing the effect for deforming the shape of hair. In contrast, from the viewpoint of enhancing touch feeling after hair deforming treatment, the treatment method preferably includes an intermediate rinsing step. When an intermediate rinsing step is included, the molecular weight of component (B) contained in the first agent is preferably 140 to 1,000, more preferably 180 to 1,000, for further enhancing the effect for deforming the shape of hair and attaining favorable touch feeling after hair deforming.

Also, when the successive application-type multi-agent type hair deforming agent is used, no particular limitation is imposed on the amounts of the first and second agents applied to hair. The molar ratio of component (B) to component (A) applied to hair, (B)/(A), is preferably 0.001 or higher, more preferably 0.1 or higher, still more preferably 0.2 or higher, and yet more preferably 0.25 or higher, and preferably lower than 2.5, more preferably 2.3 or lower, still more preferably 2.0 or lower, yet more preferably 1.5 or lower, in application of the hair deforming agent. In the present specification, each of the amounts of components (A) and (B) applied to hair is calculated from the amount of the first agent or the second agent applied to the hair, and the component (A) content or component (B) content of the first agent or the second agent.

An optional step of allowing the hair to which the hair deforming agent has been applied to stand for a specific period of time may be included between step (ii) and step (iii). In this case, for suitably causing the hair deforming agent to permeate and diffuse in hair, the time of allowing to stand is preferably 1 minute or longer, more preferably 3 minutes or longer, still more preferably 5 minutes or longer, and preferably 1 hour or shorter, more preferably 30 minutes or shorter, still more preferably 20 minutes or shorter.

In the step of allowing the hair to stand, the hair may be heated for promoting permeation of the hair deforming agent. When heating is performed, the heating temperature is preferably 40 to 90° C. Though heating, a low-molecular-weight oligomer can be polymerized in hair shafts before step (iii). Thus, step (iii) can be performed more advantageously, which is preferred.

Between step (ii) and step (iii), the hair to which the hair deforming agent has been applied may or may not be rinsed. However, no rinsing is preferably performed. In this case, the effects of sufficiently retaining components of the hair deforming agent in hair, imparting a semi-permanent shape to hair, and semi-permanently re-deforming the hair by heating can be further ensured.

Step (iii): Hair Deforming Step

For imparting a semi-permanent shape to hair and semi-permanently re-deforming the deformed hair by heat, the heating temperature in step (iii) is preferably 50° C. or higher, more preferably 60° C. or higher, still more preferably 80° C. or higher. In order to suppress rapid evaporation of water during heating, the heating temperature is preferably 250° C. or lower, more preferably 240° C. or lower, still more preferably 230° C. or lower. Examples of the heating means include a hair iron, an electrically heating rod, and a hot curler.

The heating time in step (iii) is appropriately adjusted in accordance with the apparatus and temperature. However, in order to permeate and diffuse the hair deforming agent in hair shafts to induce satisfactory polymerization, the heating time is preferably 1 second or longer, more preferably 5 seconds or longer, still more preferably 1 minute or longer, yet more preferably 5 minutes or longer, further more preferably 15 minutes or longer, further more preferably 30 minutes or longer. From the viewpoint of suppression of damage to hair, the heating time is preferably 2 hours or shorter, more preferably 1 hour or shorter, still more preferably 45 minutes or shorter.

The shaping performed in step (iii) encompasses straightening and curling. Examples of the straightening method include blow-heating the hair by means of a hair drier, while drawing the hair by means of a tool such as the hand, a comb, or a brush, and heating the hair by means of a hair iron. From the viewpoint easiness of shaping, a hair iron is preferably employed. In one mode of straightening the hair by heating with a hair iron, the hair is nipped by a flat iron, and the flat iron is caused to slide from hair roots to hair ends. In another mode, the hair is nipped by a flat iron, while drawing the hair by means of a tool such as the hand, a comb, or a brush, and the hair is maintained in that state. Needless to say, the two modes may be combined. In curling the hair, examples of the curling method include heating the hair which is wound by electrically heating rods, a hot curler, or the like for a certain period of time, and winding the hair by a curl iron for a certain period of time.

Preferably, step (iii) is performed under the conditions where rapid water evaporation is suppressed. Specific means for suppressing evaporation of water include wrapping the hair to which the hair deforming agent has been applied with a plastic film (e.g., a food wrapping film), a cap, or the like, and continuously spraying steam (e.g., superheated steam) to the hair.

After carrying out step (iii), the shaped hair may or may not be rinsed. However, rinsing is preferably performed so as to prevent impairment hair touch feeling due to remaining polymers.

### (Hair Re-Deforming Method)

After the hair has been deformed through a method including steps (ii)-(iii), the deformed hair may be subjected to a step of semi-permanently re-deforming the hair to a different shape by heating (i.e., a re-deforming step). The heating temperature in re-deforming is preferably 30° C. or higher, more preferably 40° C. or higher, and preferably 230° C. or lower, more preferably 220° C. or lower, still more preferably 210° C. or lower. In the re-deforming of hair, application of the hair deforming agent of the present invention, a hair treatment composition containing a reducing agent (e.g., a permanent wave agent), or another type of hair treatment composition such as an alkali relaxer is preferably omitted.

Hereinafter, specific procedures of a step of semi-permanently re-deforming the hair to a different shape by heating will be described.

### Re-Deforming of Shaped Curly Hair to Straight Hair

Examples of the method of re-deforming shaped curly hair to straight hair include blow-heating the hair by means of a hair drier, while drawing the hair by means of a tool such as the hand, a comb, or a brush, and heating the hair by means of a hair iron. From the viewpoint easiness of hair deforming, a hair iron is preferably employed. In one procedure of straightening the hair by heating with a hair iron, the hair is nipped by a hair iron, and the hair iron is caused to slide from hair roots to hair ends. In another procedure, the hair is nipped by a hair iron, while drawing the hair by means of a tool such as the hand, a comb, or a brush, and the hair is maintained in that state. The two procedures may be combined.

Under any conditions, including the type of the hair iron, the material of heating members thereof, the heating temperature employed, and the mode of operating the hair iron, in order to attain semi-permanent or permanent hair deforming, the actual temperature (hair temperature) during heating of the hair is preferably 120° C. or higher, more preferably 150° C. or higher. From the viewpoints of prevention of hair damage and deforming hair semi-permanently or permanently, the temperature is preferably 230° C. or lower, more preferably 220° C. or lower, still more preferably 210° C. or lower.

The temperature of heating hair can be monitored by means of, for example, a radiation thermometer (model ST653, product of SENTRY).

### Re-Deforming of Straightened Hair to Curly Hair

Examples of the method of deforming straightened hair to curly hair include heating the hair which is wound by heating rods, a hot curler, or the like for a certain period of time, and winding the hair by a hair iron for a certain period of time.

In the re-deforming method, the actual temperature (hair temperature) during heating of the hair is preferably 30° C. or higher, more preferably 40° C. or higher, in order to suitably deforming the hair permanently or semi-permanently. From the viewpoints of prevention of hair damage and deforming hair semi-permanently or permanently, the temperature is preferably 180° C. or lower, more preferably 120° C. or lower, still more preferably 100° C. or lower, yet more preferably 80° C. or lower, further more preferably 60° C. or lower.

In re-deforming of hair, heating may be performed when the hair is dry or after the hair has been wetted with water. From the viewpoint of deforming hair semi-permanently or permanently, heating is preferably performed after the hair has been wetted with water.

The time of heating hair in hair re-deforming may be appropriately tuned in accordance with the heating tool, heating temperature, and other conditions employed in heating. From the viewpoint of deforming hair semi-permanently or permanently, the heating time is preferably 1 second or longer, more preferably 5 seconds or longer, still more preferably 1 minute or longer, yet more preferably 5 minutes or longer, further more preferably 15 minutes or longer, further more preferably 30 minutes or longer. In order to prevent hair damage, the heating time is preferably 2 hours or shorter, more preferably 1 hour or shorter, still more preferably 45 minutes or shorter.

The hair treatment method of the present invention provides a technique which enables hair to have any desired shape, based on a principle completely different from that of the perming treatment using a reducing agent or the relaxing treatment employing a strong alkaline (pH 12-14) hair treatment composition. In other words, the present method does not include a step of applying a hair treatment composition containing a reducing agent or applying a strong alkaline (pH 12-14) hair treatment composition. Therefore, one advantage of the hair treatment method of the present invention, as compared with existing hair deforming methods, is that the hair can be shaped without causing damage thereto.

In relation to the above-described embodiments, there will next be disclosed preferred embodiments of the present invention.

<1>

A hair treatment method including the following steps (i) to (iii) for dyeing hair and at the same time deforming hair semi-permanently or permanently:

(i) a step of dyeing hair through a hair treatment including applying a dye to the hair,
(ii) a step of applying the following components (A), (B), and (C) to the hair,

(A): one or more compounds selected from the group consisting of glyoxylic acid, a glyoxylic acid hydrate, a glyoxylate salt, and a glyoxylamide;

(B): a phenolic compound having an electron-donating group on at least one m-position and having a hydrogen atom on at least one of the o-positions and the p-position, wherein the electron-donating group on the m-position may form, together with an adjacent carbon atom, a benzene ring optionally substituted with hydroxyl group(s);

(C): water, and

(iii) a step of heating and shaping the hair.
<2>

The hair treatment method as recited in <1> above, wherein, in step (ii), the molar ratio of component (B) to component (A) applied to hair, (B)/(A), is preferably 0.001 or higher, more preferably 0.1 or higher, still more preferably 0.2 or higher, and yet more preferably 0.25 or higher, and preferably lower than 2.5, more preferably 2.3 or lower, still more preferably 2.1 or lower, yet more preferably 1.9 or lower, yet more preferably 1.7 or lower, yet more preferably 1.6 or lower.

<3>

The hair treatment method as recited in <1> or <2> above, wherein step (i) preferably includes a step of applying a hair dye composition containing one or more dyes selected from the group consisting of direct dyes and oxidative dyes.

<4>

The hair treatment method as recited in <3>, wherein the direct dye is preferably one or more species selected from the group consisting of an anionic dye, a nitro dye, a cationic dye, one or more dyes which have azophenol structure selected from the group consisting of HC Red 18, HC Blue 18, and HC Yellow 16, and a salt thereof.

<5>

The hair treatment method as recited in <3> or <4>, wherein the method preferably includes a step of washing the hair dye composition present on the hair.

<6>

The hair treatment method as recited in any one of <1> to <5>, wherein the heating temperature in step (iii) is preferably 50° C. or higher, more preferably 60° C. or higher, still more preferably 80° C. or higher, and is preferably 250° C. or lower, more preferably 240° C. or lower, still more preferably 230° C. or lower.

<7>

The hair treatment method as recited in any one of <1> to <6>, wherein step (ii) preferably includes a step of applying the hair deforming agent containing components (A), (B), and (C) to hair.

<8>

The hair treatment method as recited in <7>, wherein the hair deforming agent is preferably a one-agent type hair deforming agent.

<9>

The hair treatment method as recited in <8>, wherein the pH of the hair deforming agent is preferably 4 or lower, more preferably 3 or lower, still more preferably 2.5 or lower, yet more preferably 2 or lower, and, preferably 1 or higher, more preferably 1.2 or higher, still more preferably 1.5 or higher.

<10>

A hair treatment method including the following steps for dyeing hair and deforming hair semi-permanently or permanently:

1) optionally wetting hair with water;

2) applying the hair deforming agent as recited in <8> or <9> to the hair;

3) optionally allowing the hair to which the hair deforming agent has been applied to stand for 1 minute or more and 1 hour or less, wherein the hair may optionally be heated at 40° C. or higher and 90° C. or lower;

4) heating and shaping the hair at 50° C. or higher and 250° C. or lower;

5) optionally rinsing the hair;

6) optionally heating and reshaping the hair at 40° C. or higher and 230° C. or lower; and

7) optionally heating and further shaping the re-deformed hair at 40° C. or higher and 230° C. or lower.

<11>

The hair treatment method as recited in <7>, wherein the hair deforming agent is a multi-agent type hair deforming agent including a first agent containing components (B) and (C), and a second agent containing components (A) and (C).

<12>

The hair treatment method as recited in <11>, wherein the pH of the second agent of the hair deforming agent is preferably 4 or lower, more preferably 3 or lower, still more preferably 2.5 or lower, yet more preferably 2 or lower, and, preferably 1 or higher, more preferably 1.2 or higher, still more preferably 1.5 or higher.

<13>

A hair treatment method including the following steps for dyeing hair and deforming hair semi-permanently or permanently:

1) optionally wetting hair with water;

2) mixing the first agent and the second agent of the hair deforming agent as recited in <11> or <12> and applying the obtained hair deforming agent to hair;

3) optionally allowing the hair to which the hair deforming agent has been applied to stand for 1 minute or more and 1 hour or less, wherein the hair may optionally be heated at 40° C. or higher and 90° C. or lower;

4) heating and shaping the hair at 50° C. or higher and 250° C. or lower;

5) optionally rinsing the hair;

6) optionally heating and reshaping the hair at 40° C. or higher and 230° C. or lower; and

7) optionally heating and further shaping the re-deformed hair at 40° C. or higher and 230° C. or lower.

<14>

A hair treatment method including the following steps for dyeing hair and deforming hair semi-permanently or permanently:

1) optionally wetting hair with water;

2) applying the first agent of the hair deforming agent as recited in <11> or <12> to hair;

3) optionally allowing the hair to stand for 1 minute or more and 1 hour or less, wherein the hair may optionally be heated at 40° C. or higher and 90° C. or lower;

4) optionally washing off the first agent present on the hair;

5) applying the second agent of the hair deforming agent as recited in <11> or <12> to a portion of the hair to which the first agent has been applied;

component (A): one or more compounds selected from the group consisting of glyoxylic acid, a glyoxylic acid hydrate, a glyoxylate salt, and a glyoxylamide;

component (C): water;

6) optionally allowing the hair to stand for 1 minute or more and 1 hour or less, wherein the hair may optionally be heated at 40° C. or higher and 90° C. or lower;

7) heating and shaping the hair at 50° C. or higher and 250° C. or lower;

8) optionally rinsing the hair;

9) optionally heating and reshaping the hair at 40° C. or higher and 230° C. or lower; and 10) optionally heating and further shaping the re-deformed hair at 40° C. or higher and 230° C. or lower.

<15>

The hair treatment method as recited in <14>, wherein the molecular weight of component (B) contained in the first agent is preferably 140 or more and 1,000 or less, more preferably 180 to 1,000

<16>

The hair treatment method as recited in any one of <1> to <15>, which method preferably does not include a step of applying, to the hair, a hair treatment composition containing a reducing agent.

<17>

The hair treatment method as recited in any one of <1> to <16>, which method preferably does not include a step of applying a strong alkaline hair treatment composition having a pH of 12 or higher and 14 or lower.

<18>

The hair treatment method as recited in any one of <1> to <17>, wherein the step of heating and shaping the hair is preferably performed under the conditions where evaporation of water is suppressed.

<19>

The hair treatment method as recited in any one of <7> to <18>, wherein the amount of component (A) contained in the hair deforming agent, relative to the total composition of the hair deforming agent and in terms of glyoxylic acid, is preferably 1 mass % or higher, more preferably 2 mass % or higher, still more preferably 2.5 mass % or higher, yet more preferably 3 mass % or higher, and preferably 30 mass % or lower, more preferably 25 mass % or lower, still more preferably 20 mass % or lower, yet more preferably 15 mass % or lower, further more preferably 12 mass % or lower.

<20>

The hair treatment method as recited in any one of <7> to <19>, wherein the amount of component (B) contained in the hair deforming agent, relative to the total composition of the hair deforming agent, is preferably 0.2 mass % or higher, more preferably 0.5 mass % or higher, still more preferably 1 mass % or higher, yet more preferably 1.5 mass % or higher, and preferably 40 mass % or lower, more preferably 30 mass % or lower, still more preferably 25 mass % or lower, yet more preferably 23 mass % or lower, further more preferably 20 mass % or lower.

<21>

The hair treatment method as recited in any one of <1> to <20>, wherein component (B) is preferably resorcin (B1).

<22>

The hair treatment method as recited in <21>, wherein the amount of component (B1) contained in the hair deforming agent, relative to the total composition of the hair deforming agent, is preferably 1 mass % or higher, more preferably 2 mass % or higher, still more preferably 3 mass % or higher, yet more preferably 4 mass % or higher, further more preferably 5 mass % or higher, and, preferably 40 mass % or lower, more preferably 30 mass % or lower, still more preferably 25 mass % or lower, yet more preferably 20 mass % or lower, further more preferably 17 mass % or lower.

<23>

The hair treatment method as recited in <21> or <22>, wherein the molar ratio (B1)/(A) of component (B1) to component (A) applied to hair is preferably 0.2 or higher, more preferably 0.3 or higher, still more preferably 0.4 or higher, yet more preferably 0.5 or higher, further more preferably 0.7 or higher, and, preferably lower than 2.5, more preferably 2.3 or lower, still more preferably 2 or lower, yet more preferably 1.5 or lower, further more preferably 1.2 or lower.

<24>

The hair treatment method as recited in any one of <1> to <20>, wherein component (B) is preferably one or more compounds (B2) represented by the following formula (1):

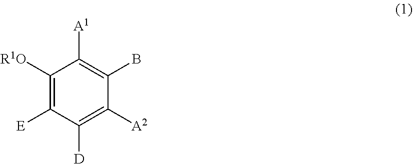

(wherein:

R¹ represents a hydrogen atom or a methyl group;

A¹ and A², which may be identical to or different from each other, each represent a hydrogen atom, a C1 to C12 linear-chain or branched-chain alkyl or alkenyl group, an optionally substituted C7 to C12 aralkyl or arylalkenyl group, a C1 to C6 linear-chain or branched-chain alkoxy or alkenyloxy group, a halogen atom, or —CO—R² (wherein R² represents a C1 to C12 linear-chain or branched-chain alkyl or alkenyl group, an optionally substituted C7 to C12 aralkyl or arylalkenyl group, or an optionally substituted C6 to C12 aromatic hydrocarbon group);

B represents a hydrogen atom, a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group);

D represents a hydrogen atom, a hydroxyl group, a methyl group, or a C1 to C12 linear-chain or branched-chain alkoxy or alkenyloxy group; and

E represents a hydrogen atom, a hydroxyl group, a C1 to C6 linear-chain or branched-chain alkyl or alkenyl group, or a C1 to C6 linear-chain or branched-chain alkoxy or alkenyloxy group,

wherein two or three of A¹, A², B, and E each are a hydrogen atom, with each of remaining being a group which is not a sulfo group, and in the case where D is a hydrogen atom or a methyl group, A¹ and B, or A² and B form an optionally hydroxyl group-substituted benzene ring with two adjacent carbon atoms), more preferably compounds represented by the following formula (1-1), (1-2), (1-3-a), or (1-3-b):

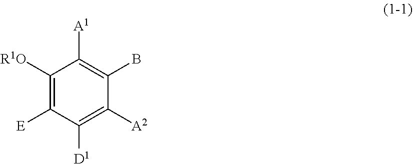

wherein R¹, A¹, A², B, and E are defined as above, and D¹ represents a hydroxyl group or a methoxy group;

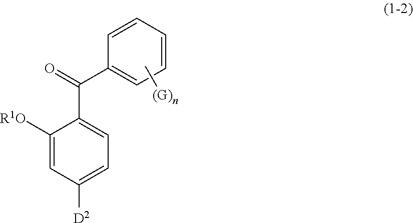

wherein R¹ has the same meaning as defined above; D² represents a hydroxyl group or a C1 to C12 alkoxy group; G represents a hydroxyl group, a C1 to C6 linear-chain or branched-chain alkyl or alkenyl group, or a C1 to C6 alkoxy group; and n is an integer of 0 to 2;

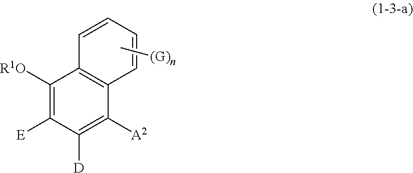

wherein R¹, A², E, D, G, and n are defined as above; or

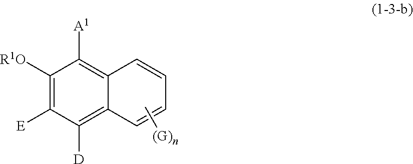

wherein R¹, A², E, D, G, and n are defined as above, more preferably one or more compounds represented by formula (1-1-1), (1-1-2), or (1-1-3):

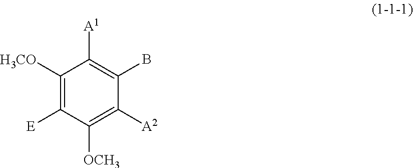

wherein A¹, A², B, and E are defined as above;

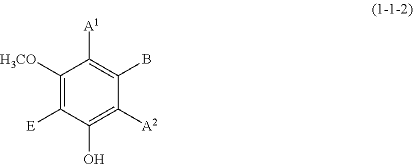

wherein A¹, A², B, and E are defined as above; or

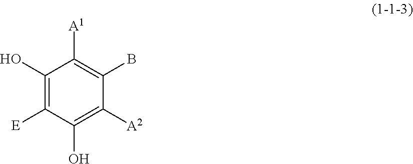

wherein A¹, A², B, and E are defined as above.
<25>

The hair treatment method as recited in <24> above, wherein component (B2) is preferably one or more species selected from the group consisting of 2-methylresorcin, 4-chlororesorcin, 4-alkylresorcin, 4-aralkylresorcin, 4-acylated resorcin, 5-alkylresorcin, 5-aralkylresorcin, 5-hydroxyarylalkenylresorcin, 2,4,6-trihydroxyphenylaralkylketone, gallic acid, and a gallate ester; more preferably 4-butylresorcin (trivial name: rucinol), 4-(1-phenylethyl)resorcin (trivial name: Symwhite 377), 5-(hydroxyphenylethenyl)resorcin (trivial name: resveratrol), 3-hydroxyphenyl-1-(benzene-2,4,6-triol)propan-1-one (trivial name: phloretin), 4-(2,4-dihydroxybenzoyl)resorcin (trivial name: Benzophenone-2), 5-(hydroxyphenylethenyl)-1,3-dimethoxybenzene (trivial name: pterostilbene), and 1-naphthol; still more preferably one or more species selected from the group consisting of 2-methylresorcin, 4-chlororesorcin, 1-naphthol, 4-n-butylresorcinol, 4-phenylresorcinol, 5-(hydroxyphenylethenyl) resorcin, 3-hydroxyphenyl-1-(benzene-2,4,6-triol)propan-1-one, and 4-(2,4-dihydroxybenzoyl)resorcin.

<26>

The hair treatment method as recited in <24> or <25>, wherein the amount of component (B2) contained in the hair deforming agent, relative to the total hair deforming agent, is preferably 0.2 mass % or higher, more preferably 0.5 mass % or higher, still more preferably 1 mass % or higher, yet more preferably 5 mass % or higher, further more preferably 10 mass % or higher, and, preferably 40 mass % or lower, more preferably 30 mass % or lower, still more preferably 25 mass % or lower, yet more preferably 23 mass % or lower, further more preferably 20 mass % or lower.

<27>

The hair treatment method as recited in any one of <24> to <26>, wherein the molar ratio (B2)/(A) of component (B2) to component (A) applied to hair is preferably 0.1 or higher, more preferably 0.3 or higher, still more preferably 0.5 or higher, yet more preferably 0.7 or higher, and, preferably lower than 2.5, more preferably 2.3 or lower, still more preferably 2 or lower, yet more preferably 1.5 or lower, further more preferably 1.2 or lower.

<28>

The hair treatment method as recited in <1> to <20>, wherein component (B) is preferably one or more compounds (B3) represented by the following formula (2):

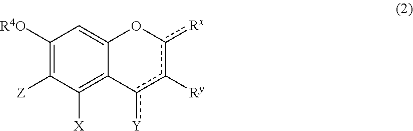

wherein,

R⁴ represents a hydrogen atom or a methyl group;

X represents a hydrogen atom, a hydroxyl group, or a methoxy group;

Y represents a hydrogen atom, an oxygen atom, a hydroxyl group, or a methoxy group;

Z represents a hydrogen atom or a C1 to C5 linear-chain or branched-chain alkyl or alkenyl group;

R^{x} represents a hydrogen atom, an oxygen atom, a hydroxyl group, a methoxy group, or an aromatic hydrocarbon group which may be substituted with 1 to 3 hydroxyl groups or methoxy groups and which optionally forms a condensed ring with 1,3-dioxolane;

R^{y} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups;

a dotted line may be a double bond;

each of the combinations of the dotted line and the solid line being adjacent to R^{x} or Y denotes a double bond in the case where R^{x} or Y is an oxygen atom, and denotes a single bond in the other cases; and

Z represents a C1 to C5 linear-chain or branched-chain alkyl or alkenyl group, only when R^{x} or R^{y} is an o,p-dihydroxyaromatic hydrocarbon group, and represents a hydrogen atom in the other cases, more preferably one or more compounds represented by the following formula (2-1), (2-2), (2-3), (2-4), or (2-5);

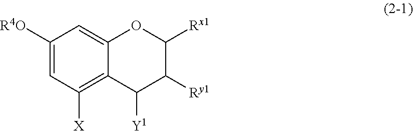

R⁴ and X are defined as above;

Y¹ represents a hydrogen atom, a hydroxyl group, or a methoxy group;

R^{x1} represents an aromatic hydrocarbon group which may be substituted with 1 to 3 hydroxyl groups or methoxy groups and which optionally forms a condensed ring with 1,3-dioxolane; and

R^{y1} represents a hydrogen atom, a hydroxyl group, a methoxy group, an aromatic hydrocarbon group which may be substituted with 1 to 3 hydroxyl groups or methoxy groups and which optionally forms a condensed ring with 1,3-dioxolane, or an arylcarbonyloxy or aralkylcarbonyloxy group which may be substituted with 1 to 3 hydroxyl groups or methoxy groups;

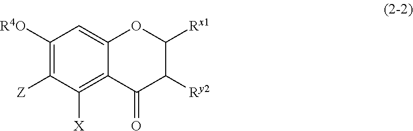

wherein R⁴, X, Z, and R^{x1} are defined as above, and R^{y2} represents a hydrogen atom, a hydroxyl group, or a methoxy group; and

Z represents a C1 to C5 linear-chain or branched-chain alkyl or alkenyl group, only when R^{x1} is an o,p-dihydroxyaromatic hydrocarbon group, and represents a hydrogen atom in the other cases;

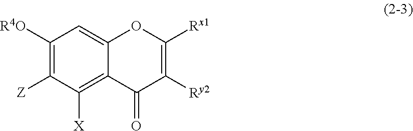

wherein R⁴, X, Z, R^{x1}, and R^{y2} are defined as above, and

Z is a C1 to C5 linear-chain or branched-chain alkyl or alkenyl group, only when R^{x1} is an o,p-dihydroxyaromatic hydrocarbon group. In the other cases, Z is a hydrogen atom;

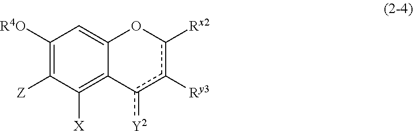

wherein

R⁴, X, Z, and the dotted line portion are defined as above;

Y² represents a hydrogen atom or an oxygen atom;

R^{x2} represents a hydrogen atom, a hydroxyl group, or a methoxy group;

R^{y3} represents an aromatic hydrocarbon group which may be substituted with 1 to 3 hydroxyl groups or methoxy groups and which optionally forms a condensed ring with 1,3-dioxolane;

the combination of the dotted line and the solid line being adjacent to Y² denotes a double bond in the case where Y² is an oxygen atom, and denotes a single bond in the other cases; and

Z represents a C1 to C5 linear-chain or branched-chain alkyl or alkenyl group, only when R^{y3} is an o,p-dihydroxyaromatic hydrocarbon group, and represents a hydrogen atom in the other cases; or

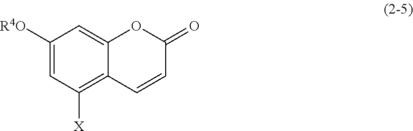

wherein R⁴ and X are defined as above.
<29>

The hair treatment method as recited in <28>, wherein component (B3) is preferably one or more compounds selected from the group consisting of catechin, epicatechin, epigallocatechin, catechin gallate, epicatechin gallate, epigallocatechin gallate, quercetin, morin, hesperetin, naringenin, chrysin, daidzein, equol, and umbelliferone.

<30>

The hair treatment method as recited in <28> or <29>, wherein the component (B3) content, relative to the total composition of the hair deforming agent, is preferably 0.2 mass % or higher, more preferably 0.5 mass % or higher, still more preferably 1 mass % or higher, yet more preferably 1.5 mass % or higher, and from the viewpoint of suitably mixing components, preferably 30 mass % or lower, more preferably 25 mass % or lower, yet more preferably 23 mass % or lower, further more preferably 20 mass % or lower.

<31>

The hair treatment method as recited in any one of <28> to <30>, wherein the molar ratio (B3)/(A) of component (B3) to component (A) applied to hair is preferably 0.01 or higher, more preferably 0.1 or higher, still more preferably 0.3 or higher, yet more preferably 0.5 or higher, and, preferably lower than 2.5, more preferably 2.3 or lower, still more preferably 2 or lower, yet more preferably 1.5 or lower.

<32>

A hair treatment method including the following steps (i) to (iii) for dyeing hair and at the same time deforming hair semi-permanently or permanently:

(i) a step of dyeing hair through a hair treatment including applying one or more dye(s) selected from the group consisting of an anionic dye, a nitro dye, a cationic dye, one or more dyes which have azophenol structure selected from the group consisting of HC Red 18, HC Blue 18, and HC Yellow 16, and a salt thereof to the hair,
(ii) a step of applying a hair deforming agent comprising the following components (A), (B1), and (C) to the hair,

(A): one or more compounds selected from the group consisting of glyoxylic acid, a glyoxylic acid hydrate, a glyoxylate salt, and a glyoxylamide;

(B1): 5 mass % or higher and 40 mass % or lower of resorcin,

(C): water, and

(iii) a step of heating and shaping the hair at 50° C. or higher and 250° C.
<33>

A hair treatment method including the following steps (i) to (iii) for dyeing hair and at the same time deforming hair semi-permanently or permanently:

(i) a step of dyeing hair through a hair treatment including applying one or more dye(s) selected from the group consisting of an anionic dye, a nitro dye, a cationic dye, one or more dyes which have azophenol structure selected from the group consisting of HC Red 18, HC Blue 18, and HC Yellow 16, and a salt thereof to the hair,
(ii) a step of applying a hair deforming agent comprising the following components (A), (B2), and (C) to the hair,

(A): one or more compounds selected from the group consisting of glyoxylic acid, a glyoxylic acid hydrate, a glyoxylate salt, and a glyoxylamide;

(B2): 5 mass % or higher and 30 mass % or lower of one or more phenolic compound(s) selected from the group consisting of 2-methylresorcin, 4-butylresorcin (trivial name: Rucinol), 4-hexylresorcin, 4-(1-phenylethyl)resorcin (trivial name: Symwhite377), 4-chlororesorcin, 5-(hydroxyphenylethenyl)resorcin (trivial name: resveratrol), 5-(hydroxyphenylethenyl)-1,3-dimethoxybenzene (trivial name: Pterostilbene), 3-hydroxyphenyl-1-(benzene-2,4,6-triol)propane-1-on (trivial name: Phloretin), 4-(2,4-dihydroxybenzoyl)resorcin (trivial name: Benzophenone-2) and 1-naphthol,

(C): water, and

(iii) a step of heating and shaping the hair at 50° C. or higher and 250° C.
<34>

A hair treatment method including the following steps (i) to (iii) for dyeing hair and at the same time deforming hair semi-permanently or permanently:

(i) a step of dyeing hair through a hair treatment including applying one or more dye(s) selected from the group consisting of an anionic dye, a nitro dye, a cationic dye, one or more dyes which have azopheol structure selected from the group consisting of HC Red 18, HC Blue 18, and HC Yellow 16, and a salt thereof to the hair,
(ii) a step of applying a hair deforming agent comprising the following components (A), (B3), and (C) to the hair,

(A): one or more compounds selected from the group consisting of glyoxylic acid, a glyoxylic acid hydrate, a glyoxylate salt, and a glyoxylamide;

(B3): 5 mass % or higher and 30 mass % or lower of one or more phenolic compound(s) selected from the group consisting of catechin, epigallocatechin, epigallocatechin gallate, naringenin and equol,

(C): water, and

(iii) a step of heating and shaping the hair at 50° C. or higher and 250° C.
<35>

The hair treatment method as described in any one of <32> to <34>, wherein the amount of hair reducing agent in the hair deforming agent is preferably 0.1 mass % or lower, more preferably the agent contains substantially no hair reducing agent.

### EXAMPLES

### Example 1

A two-agent-type hair dye composition and a one-agent type hair deforming agent, shown in Table 1, were prepared and used in combination (Example 1). Shape-giving effect and dyeing performance were evaluated through the following procedure and on the basis of the following criteria. The results are also shown in Table 1. The pH of each composition was obtained from a sample of the composition which was not diluted after preparation and was measured at room temperature (25° C.) by means of a pH meter (model F-52, product of HORIBA).

### <Shape-Giving Effect>

1. The first agent of the hair dyeing agent and the second agent of the hair dyeing agent, shown in Table 1, were mixed at a mass ratio of 1:1.

2. The mixture obtained in 1. above was applied to a 25-cm tress (0.5 g) of Caucasian straight hair (untreated) at a bath ratio by mass of 1:2, and distributed evenly.

3. The hair tress was wrapped with aluminum foil, and placed in a 40° C. oven to heat for 30 minutes.

4. The hair tress was taken out of the oven and cooled to a room temperature.

5. The sample was rinsed with a flow of tap water at 30° C. for 30 seconds. Thereafter, a shampoo for assessment was lathered on the tress for 60 seconds, and was then rinsed with running tap water at 30° C. for 30 seconds.

6. The hair tress was dried with cool air from a drier for 5 minutes while being combed.

7. The hair tress was wetted with tap water at 30° C. for 30 seconds, and the wetted hair tress was wound around a plastic rod (diameter: 14 mm), followed by fixing with a clip.

8. Onto the thus-wound hair tress, a hair deforming agent (1 g) was applied, and the hair tress wound over the rod was tightly wrapped with plastic film. The sample was heated in an oven at 90° C. for 1 hour.

9. The hair tress was removed from the oven and cooled to a room temperature.

10. The tress was removed from the rod, and was then rinsed with running tap water at 30° C. for 30 seconds. Thereafter, a shampoo for assessment was lathered on the tress for 60 seconds, and was then rinsed with running tap water at 30° C. for 30 seconds.

11. The tress was immersed at an infinite bath ratio in tap water at 30° C. for 60 seconds. Thereafter, the tress was gently pulled up out of the water while holding the root thereof, and water was then drained off by lightly shaking it.

12. The hair tress was suspended in a laboratory and allowed to stand for 2 hours to dryness. Then the hair tress was combed and the length of the perpendicularly suspended sample (i.e., the distance from the root of the sample to a point most distant therefrom) was measured.

The percent decrease in tress length (I, %), obtained from the length of the untreated tress sample (L₀) and the length of the treated tress sample (L) by the following equation was defined as the curl tightness. The sample was evaluated on the basis of curl tightness. The sample was classified into five grades A through E on the basis of the following criteria.

I=[(L₀−L)/L₀]×100

### A: 40≤I

### B: 20≤I<40

### C: 10≤I<20

### D: 2.5≤I<10

### E: I<2.5

### <Dyeing Performance>

1. A 10-cm tress of white hair (untreated) (1.0 g) was dyed following steps 1 to 6 of the shape-giving effect test. Subsequently, the hair tress was wetted with tap water at 30° C. for 30 seconds, and the water was lightly removed by a towel.

2. The hair tress was placed on a piece of plastic film, and the hair deforming agent (1.5 g) was applied and distributed evenly. After that, another piece of plastic film was placed from above to cover the tress, and was fixed using mending tape such that the tress was sealed in a sack-shaped space. This was heated in an oven at 90° C. for 1 hour.

3. The tress sealed by the pieces of plastic film was removed from the oven and cooled to a room temperature.

4. The tress was removed from the plastic film and rinsed with a flow of tap water at 30° C. for 30 seconds. Thereafter, a shampoo for assessment was lathered on the tress for 60 seconds. Subsequently, the tress was rinsed with a flow of tap water at 30° C. for 30 seconds. After that, the tress was dried with cool air from a drier for 5 minutes while being combed.

5. The colors of the front and back sides of the tress were measured at the vicinity of the root, intermediate, and end portion of the tress through use of a colorimeter (colorimeter CR-400, product of Konica Minolta, Inc.). The average of colorimetric values at 6 points in total was employed as a measured colorimetric value (L, a, b).

6. The degree of dyeing was evaluated by ΔE*ab, using as a reference the case which omitted the dyeing treatment of steps 1 to 6 described in the shape-giving effect test. The color measurement was performed on the day on which the tress was dyed.

### (Formulation of Shampoo for Assessment)

| Components | (mass %) |
|---|---|
| Sodium laureth sulfate | 15.5 |
| Lauramide DEA | 1.5 |
| Sodium benzoate | 0.5 |
| EDTA-2Na | 0.3 |
| Phosphoric acid | amount to pH 7 |
| Deionized water | balance |
| Total | 100 |

TABLE 1

| | | | | Example |
|---|---|---|---|---|
| | | | | 1 |
| Dyeing | First dyeing | Cetearyl alcohol | | 10.8 |
| treatment | agent | Oleth-5 | | 5.0 |
| (pre- | Formulation | Oleic acid | | 2.5 |
| treatment) | [mass %] | Cocamide MEA | | 4.6 |
| | | Sodium lauryl sulfate | | 1.7 |
| | | propylene glycol monostearate | | 0.6 |
| | | Na sulfite anhydrate | | 0.5 |
| | | Aqueous ammonia (28 mass %) | | 6.5 |
| | | Toluene 2,5-diaminesulfate | | 1.3 |
| | | Resorcin | | 0.5 |
| | | m-Aminophenol | | 0.2 |
| | | 2,4-diaminophenoxyethanol | | 0.02 |
| | | hydrochloride | | |
| | | Purified water | | bal |
| | Second dyeing | Aq. hydrogen peroxide (35 mass %) | | 17.1 |
| | agent | Cetearyl alcohol | | 1.7 |
| | Formulation | Sodium lauryl sulfate | | 0.2 |
| | [mass %] | Phosphoric acid | | 0.3 |
| | | Salicylic acid | | 0.01 |
| | | Purified water | | bal |
| | Hair deforming | (B) | Resorcin | 30 |
| | agent | (A) | Glyoxylic acid | 20 |
| | Formulation | — | Sodium hydroxide | * |
| | [mass %] | (C) | Purified water | bal |

| | |
|---|---|
| Total | 100 |

| | | |
|---|---|---|
| | pH | 2.0 |
| Shape- | Curl tightness | 16.5 |
| givinging | (decrease in tress length (%)) | |
| | Shape-giving effect | C |
| Dyeing | ΔE*ab | 73.2 |
| performance | | |

*: Amount for adjusting pH

### Examples 2 and 3

Two-agent-type hair dye compositions and two-agent-type hair deforming agents shown in Table 2 were prepared, and used in combination (Examples 2 and 3). Shape-giving effect and dyeing performance were evaluated through the following procedure and on the basis of the following criteria. The results are also shown in Table 2.

### <Shape-Giving Effect>

1. The first agent of the hair dyeing agent and the second agent of the hair dyeing agent, shown in Table 2, were mixed at a mass ratio of 1:1.

2. The mixture obtained in 1. above was applied to a 25-cm tress (0.5 g) of Caucasian straight hair (untreated) at a bath ratio by mass of 1:2, and distributed evenly.

3. The hair tress was wrapped with aluminum foil, and placed in a 40° C. oven to heat for 30 minutes.

4. The hair tress was taken out of the oven and cooled to a room temperature.

5. The hair tress was rinsed with a flow of tap water at 30° C. for 30 seconds, then a shampoo for assessment was lathered on the tress for 60 seconds, followed by rinsing with a flow of tap water at 30° C. for 30 seconds.

6. The hair tress was dried with cool air from a drier for 5 minutes while being combed.

7. The hair tress was wetted with tap water at 30° C. for 30 seconds, and the wetted hair tress was wound around a plastic rod (diameter: 14 mm), followed by fixing with a clip.

8. Onto the thus-wound hair tress, 1 g of the first agent of a hair deforming agent was applied, and the hair tress wound over the rod was tightly wrapped with plastic film. The sample was heated in an oven at 90° C. for 1 hour.

9. The hair tress was removed from the oven and cooled to a room temperature.

10. The hair tress was removed from the rod and rinsed with a flow of tap water at 30° C. for 30 seconds. Then, to shampoo for assessment was lathered on the tress for 60 second, followed by rinsing with a flow of tap water at 30° C. for 30 seconds.

11. The wet hair tress was wound around a plastic rod (diameter: 14 mm) and fixed with a clip.

12. Onto the thus-wound hair tress, 1 g of the second agent of the hair deforming agent was applied, and the hair tress wound over the rod was tightly wrapped with plastic film. The sample was heated in an oven at 90° C. for 1 hour.

13. The hair tress was removed from the oven and cooled to a room temperature.

14. The hair tress was removed from the rod and rinsed with a flow of tap water at 30° C. for 30 seconds. Then, a shampoo for assessment was lathered on the tress for 60 seconds.

15. The hair tress was rinsed again with a flow of tap water at 30° C. for 30 seconds and then the tress was immersed at an infinite bath ratio in tap water at 30° C. for 60 seconds. Subsequently, the tress was gently pulled up out of the water while holding the root thereof, and water was then drained off by lightly shaking it.

16. The hair tress was suspended in a laboratory and allowed to stand for 2 hours to dryness. Then the hair tress was combed and the length of the perpendicularly suspended sample (i.e., the distance from the root of the sample to a point most distant therefrom) was measured.

The percent decrease in tress length (I, %), obtained from the length of the untreated tress sample (L₀) and the length of the treated tress sample (L) by the following equation was defined as the curl tightness. The sample was evaluated on the basis of curl tightness. The sample was classified into five grades A through E on the basis of the following criteria.

I=[(L₀−L)/L₀]×100

### A: 40≤I

### B: 20≤I<40

### C: 10≤I<20

### D: 2.5≤I<10

### E: I<2.5

### <Dyeing Performance>

1. A 10-cm tress of white hair (untreated) (1.0 g) was dyed following steps 1 to 6 of the shape-giving effect test. Subsequently, the hair tress was wetted with tap water at 30° C. for 30 seconds, and the water was lightly removed by a towel.

2. The hair tress was placed on a piece of plastic film, and the first agent of the hair deforming agent (1.5 g) was applied and distributed evenly. After that, another piece of plastic film was placed from above to cover the tress, and was fixed using mending tape such that the tress was sealed in a sack-shaped space. This was heated in an oven at 90° C. for 1 hour.

3. The tress sealed by the pieces of plastic film was removed from the oven and cooled to a room temperature.

4. The tress was removed from the plastic film and rinsed with a flow of tap water at 30° C. for 30 seconds. Then, a shampoo for assessment was lathered on the tress for 60 seconds. Subsequently, the tress was rinsed with a flow of tap water at 30° C. for 30 seconds. After that, the tress was dried with cool air from a drier for 5 minutes while being combed.

5. The hair tress was placed on a piece of plastic film, and the second agent of the hair deforming agent (1.5 g) was applied and distributed evenly. After that, another piece of plastic film was placed from above to cover the tress, and was fixed using mending tape such that the tress was sealed in a sack-shaped space. This was heated in an oven at 90° C. for 1 hour.

6. The tress sealed by the pieces of plastic film was removed from the oven and cooled to a room temperature.

7. The tress was removed from the plastic film and rinsed with a flow of tap water at 30° C. for 30 seconds. Then, a shampoo for assessment was lathered on the tress for 60 seconds. Subsequently, the tress was rinsed with a flow of tap water at 30° C. for 30 seconds. After that, the tress was dried with cool air from a drier for 5 minutes while being combed.

8. The colors of the front and back sides of the tress were measured at the vicinity of the root, intermediate, and end portion of the tress through use of a colorimeter (colorimeter CR-400, product of Konica Minolta, Inc.). The average of colorimetric values at 6 points in total was employed as a measured colorimetric value (L, a, b).

9. The degree of dyeing was evaluated by ΔE*ab, using as a reference the case which omitted the dyeing treatment of steps 1 to 6 described in the shape-giving effect test. The color measurement was performed on the day on which the tress was dyed.

For the shampoo for assessment, the above-described one was used.

TABLE 2

| | | | | Example | |
|---|---|---|---|---|---|
| | | | | 2 | 3 |
| Dyeing | First dyeing | Cetearyl alcohol | | 10.8 | 10.8 |
| treatment | agent | Oleth-5 | | 5.0 | 5.0 |
| (pre-treatment) | Formulation | Oleic acid | | 2.5 | 2.5 |
| | [mass %] | Cocamide MEA | | 4.6 | 4.6 |
| | | Sodium lauryl sulfate | | 1.7 | 1.7 |
| | | propylene glycol monostearate | | 0.6 | 0.6 |
| | | Na sulfite anhydrate | | 0.5 | 0.5 |
| | | Aqueous ammonia (28 mass %) | | 6.5 | 6.5 |
| | | Toluene 2,5-diaminesulfate | | 1.3 | 1.3 |
| | | Resorcin | | 0.5 | 0.5 |
| | | m-Aminophenol | | 0.2 | 0.2 |
| | | 2,4-diaminophenoxyethanol | | 0.02 | 0.02 |
| | | hydrochloride | | | |
| | | Purified water | | bal | bal |
| | Second | Aq. hydrogen peroxide (35 mass %) | | 17.1 | 17.1 |
| | dyeing | Cetearyl alcohol | | 1.7 | 1.7 |
| | agent | Sodium lauryl sulfate | | 0.2 | 0.2 |
| | Formulation | Phosphoric acid | | 0.3 | 0.3 |
| | [mass %] | Salicylic acid | | 0.01 | 0.01 |
| | | Purified water | | bal | bal |
| Hair deforming | First hair | (B) | 4-Phenylethylresorcin | 20 | — |
| | deforming | | (+)-Catechin | — | 20 |
| | agent | — | Ethanol | 40 | 40 |
| | formulation | | HCl or NaOH | * | * |
| | [mass %] | (C) | Purified water | bal | bal |
| | | Total | | 100 | 100 |
| | pH | | | 4.0 | 4.0 |
| | Second hair | (A) | Glyoxylic acid | 20 | 20 |
| | deforming | — | Sodium hydroxide | * | * |
| | agent | (C) | Purified water | bal | bal |

| | | | |
|---|---|---|---|
| formulation | Total | 100 | 100 |
| [mass %] | | | |

| | | | |
|---|---|---|---|
| | pH | 2.0 | 2.0 |
| Shape-giving | Curl tightness | 42.0 | 26.4 |
| effect | (decrease in tress length (%)) | | |
| | Shape-giving effect | A | B |
| Dyeing | ΔE*ab | 38.2 | 39.4 |
| performance | | | |

*: Amount for adjusting pH

### Example 4 and Comparative Example 1

One-agent-type hair dye compositions and two-agent-type hair deforming agents, shown in Table 3, were prepared, and used in combination (Example 4 and Comparative Example 1). Dyeing performance and color fastness were evaluated through the following procedure and on the basis of the following criteria. The results are also shown in Table 3. Concerning Example 4, shape-giving effect was separately evaluated.

### <Shape-Giving Effect>

As Example 4, shape-giving effect was evaluated as described below.

1. Hair dye compositions shown in Table 3 were prepared.

2. A hair dye composition obtained from 1. above was applied to a 10-cm tress (1.0 g) of white hair (untreated) at a bath ratio by mass of 1:2, and distributed evenly.

3. The hair tress was wrapped with plastic film, and placed in a 40° C. oven to heat for 30 minutes.

4. The hair tress was taken out of the oven, cooled to a room temperature, and the film was removed.

5. The hair tress was rinsed with a flow of tap water at 30° C. for 30 seconds, then a shampoo for assessment was lathered on the tress for 60 seconds, followed by rinsing with a flow of tap water at 30° C. for 30 seconds.

6. The hair tress was dried with cool air from a drier for 5 minutes while being combed.

7. The hair tress was rinsed with a flow of tap water at 30° C. for 30 seconds, then a shampoo for assessment was lathered on the tress for 60 seconds, followed by rinsing with a flow of tap water at 30° C. for 30 seconds.

8. The hair tress was dried with cool air from a drier for 5 minutes while being combed.

9. The hair tress was wetted with tap water at 30° C. for 30 seconds, and the wetted hair tress was wound around a plastic rod (diameter: 14 mm), followed by fixing with a clip.

10. Onto the thus-wound hair tress, 1 g of the first agent of a hair deforming agent was applied, and the hair tress wound over the rod was tightly wrapped with plastic film. The sample was heated in an oven at 90° C. for 1 hour.

11. The hair tress was removed from the oven and cooled to a room temperature.

12. The hair tress was removed from the rod and rinsed with a flow of tap water at 30° C. for 30 seconds. Then, a shampoo for assessment was lathered on the tress for 60 seconds, followed by rinsing with a flow of tap water at 30° C. for 30 seconds.

13. The wet hair tress was wound around a plastic rod (diameter: 14 mm) and fixed with a clip.

14. Onto the thus-wound hair tress, 1 g of the second agent of the hair deforming agent was applied, and the hair tress wound over the rod was tightly wrapped with plastic film. The sample was heated in an oven at 90° C. for 1 hour.

15. The hair tress was removed from the oven and cooled to a room temperature.

16. The hair tress was removed from the rod and rinsed with a flow of tap water at 30° C. for 30 seconds. Then, then a shampoo for assessment was lathered on the tress for 60 seconds.

17. The hair tress was rinsed again with a flow of tap water at 30° C. for 30 seconds and was immersed at an infinite bath ratio in tap water at 30° C. for 60 seconds. Subsequently, the tress was gently pulled up out of the water while holding the root thereof, and water was then drained off by lightly shaking it.

18. The hair tress was suspended in a laboratory and allowed to stand for 2 hours to dryness. Then, the hair tress was combed and the length of the perpendicularly suspended sample (i.e., the distance from the root of the sample to a point most distant therefrom) was measured. It was confirmed that preferable curl shape was given to the hair tress.

### <Dyeing Performance>

As Example 4, hair tresses for assessment were prepared through the following procedure.

1. A 10-cm tress of white hair (untreated) (1.0 g) was dyed following steps 1 to 6 of the shape-giving effect test. Subsequently, the hair tress was wetted with tap water at 30° C. for 30 seconds, and the water was lightly removed by a towel.

2. The hair tress was placed on a piece of plastic film, and the first agent of the hair deforming agent (1.5 g) was applied and distributed evenly. After that, another piece of plastic film was placed from above to cover the tress, and was fixed using mending tape such that the tress was sealed in a sack-shaped space. This was heated in an oven at 90° C. for 1 hour.

3. The tress sealed by the pieces of plastic film was removed from the oven and cooled to a room temperature.

4. The tress was removed from the plastic film and rinsed with a flow of tap water at 30° C. for 30 seconds. Then, a shampoo for assessment was lathered on the tress for 60 seconds. Subsequently, the tress was rinsed with a flow of tap water at 30° C. for 30 seconds. After that, the tress was dried with cool air from a drier for 5 minutes while being combed.

5. The hair tress was placed on a piece of plastic film, and the second agent of the hair deforming agent (1.5 g) was applied and distributed evenly. After that, another piece of plastic film was placed from above to cover the tress, and was fixed using mending tape such that the tress was sealed in a sack-shaped space. This was heated in an oven at 90° C. for 1 hour.

6. The tress sealed by the pieces of plastic film was removed from the oven and cooled to a room temperature.

7. The tress was removed from the plastic film and rinsed with a flow of tap water at 30° C. for 30 seconds. Then, a shampoo for assessment was lathered on the tress for 60 seconds. Subsequently, the tress was rinsed with a flow of tap water at 30° C. for 30 seconds. After that, the tress was dried with cool air from a drier for 5 minutes while being combed.

For Comparative Example 1, hair samples were prepared as follows. Hair tresses of white hair (untreated), each having a length of 10 cm (1.0 g), first underwent the procedure of steps 2 to 7 and then the dyeing treatment of steps 1 to 6 described in the shape-giving effect test above was performed.

Through visual observation of the samples from Example 4 and Comparative Example 1, both were confirmed to have been dyed well.

For the shampoo for assessment, the above-described one was used.

### <Fastness>

A fastness test was performed on the hair tresses that had undergone the procedure of steps 1 to 7 of the dyeing performance test (Example 4 and Comparative Example 1).

1. Deionized water (40 g) was poured into a plastic bottle (Sampler (R) PP wide-mouthed bottle (50 ml)), the tress of the white hair used for the dyeing assessment was immersed into the water, and a cap was attached to the bottle.

2. The bottle including the water and the tress was placed in an oven of 40° C. for heating for 84 hours.

3. The bottle was removed from the oven and cooled to a room temperature.

4. The cap was removed from the bottle, and the white hair tress was removed. The amount of the dye leaked from the tress was evaluated on the basis of the degree of dyeing of the water in the bottle.

The assessment was conducted on the basis of the following criteria.

A. The water was as clear as the deionized water, and the opposite side of the container was clearly seen.

B. Although the water was colored, the colored water did not hinder viewing of the opposite side of the container.

C. The water was colored, and the opposite side of the container was slightly invisible.

D. The color of the water was dark, and the opposite side of the container was almost invisible.

E. The color of the water was very dark, and the opposite side of the container was completely invisible.

TABLE 3

| | | | | | Comp. |
|---|---|---|---|---|---|
| | | | | Example | Ex. |
| | | | | 4 | 1 |
| Dyeing | Dyeing | Acid Red 33 | | 0.5 | |
| treatment | agent | Hydrochloric acid | | * | |
| (pre-treatment) | Formulation | Purified water | | bal | |
| | [mass %] | Total | | 100 | |
| | | pH | | 4.0 | |
| Hair deforming | First hair | (B) | (+)-Catechin | 10 | 10 |
| | deforming | — | Ethanol | 20 | 20 |
| | agent | | HCl or NaOH | * | * |
| | formulation | (C) | Purified water | bal | bal |
| | [mass %] | Total | | 100 | 100 |
| | pH | | | 4.0 | 4.0 |
| | Second hair | (A) | Glyoxylic acid | 10 | 10 |
| | deforming | — | Sodium hydroxide | * | * |
| | agent | (C) | Purified water | bal | bal |
| | formulation | | | | |

| | [mass %] | Total | 100 | 100 |
|---|---|---|---|---|
| | pH | | 2.0 | 2.0 |
| Dyeing | Dyeing | Acid Red 33 | | 0.5 |
| treatment | agent | Hydrochloric acid | | * |
| (post- | Formulation | Purified water | | bal |
| treatment) | [mass %] | Total | | 100 |

| | | | |
|---|---|---|---|
| | pH | | 4.0 |
| Dyeing | Visual assessment | good | good |
| performance | | | |
| Fastness | Visual assessment | A | D |

*: Amount for adjusting pH

### Example 5

Each hair sample is rinsed with water and treated with a hair dye composition (direct dye composition).

Immediately thereafter, to the hair sample treated with a shampoo beforehand, the first agent and the second agent of the hair deforming agent of Example 2 are successively applied such that the mass ratio thereof is about 1:1. The agents are distributed in the hair and left on the hair for about 30 minutes. Next, the hair is dried with a hair dryer. Subsequently, the hair is deformed using a straightening iron at a temperature of about 200° C.

Hair dye composition (direct dye composition)

(Mass %)

1.50—amodimethicone

1.40—hydroxyethylcellulose

0.30—fragrance/perfume

0.55—Acid Red 52/C.I. 45100

0.28—Basic Red 51

0.01—HC Red 3

Amount to make 100 mass %—water

### Example 6

A hair dye composition (oxidative hair dye composition) is mixed with 6 mass % hydrogen peroxide at a mass ratio of 1:1, and the mixture is applied to hair. Next, similar to the method described in Example 5, the hair is deformed.

### Hair Dye Composition (Oxidative Hair Dye Composition)

(Mass %)

0.25—ammonium chloride

0.80—monoethanolamine

0.30—fragrance/perfume

0.55—p-toluenediamine

0.28—resorcinol

0.01—m-phenylenediamine

Amount to make 100 mass %—water

pH:6.8

The hair dyed and straightened by the use of the compositions described in Example 5 and 6 exhibit a good color durability, and the period during which the straight hair is kept after repeated shampooing is prolonged. In addition, the hair can be reshaped to a curl shape by the application of heat, and the curl shape after reshaping is maintained after repeated shampooing.

### Example 7

### <Wash Fastness>

Each hair sample is subjected to the procedure of steps 1 to 4 described in <dyeing performance> of Example 1, or the procedure of steps 1 to 7 described in <dyeing performance> of Examples 2 and 3, and the following test is performed to check wash fastness.

1. Each hair tress is immersed in a sodium laureth sulfate solution (5 mass %, 40 mL) whose pH has been adjusted to about 5.6 by use of citric acid, and maintained in a 30° C. bath for 24 hours.

2. The hair tress is taken out of the bath, and the amount of dye is evaluated based on the color level of shampoo water.

1. A hair treatment method comprising the following steps (i) to (iii) for dyeing hair and at the same time deforming the shape of hair semi-permanently or permanently:
(i) a step of dyeing hair through a hair treatment including applying a dye to the hair,
(ii) a step of applying the following components (A), (B), and (C) to the hair,
(A): one or more compounds selected from the group consisting of glyoxylic acid, a glyoxylic acid hydrate, a glyoxylate salt, and a glyoxylamide;
(B): one or more phenolic compounds selected from component (B1), (B2) and (B3):
(B1): resorcin;
(B2): a compound of formula (1) as defined below;
(B3): a compound of formula (2) as defined below; and
(C): water, and
(iii) a step of heating and shaping the hair: 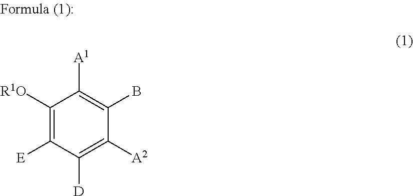
wherein:
R¹ represents a hydrogen atom or a methyl group;
A¹ and A², which may be identical to or different from each other, each represent a hydrogen atom, a C1 to C12 linear-chain or branched-chain alkyl or alkenyl group, an optionally substituted C7 to C12 aralkyl or arylalkenyl group, a C1 to C6 linear-chain or branched-chain alkoxy or alkenyloxy group, a halogen atom, or —CO—R² (wherein R² represents a C1 to C12 linear-chain or branched-chain alkyl or alkenyl group, an optionally substituted C7 to C12 aralkyl or arylalkenyl group, or an optionally substituted C6 to C12 aromatic hydrocarbon group);
B represents a hydrogen atom, a C1 to C12 linear-chain or branched-chain alkyl or alkenyl group, an optionally substituted C7 to C12 aralkyl or arylalkenyl group, or —OR³ or —COOR³ (wherein R³ represents a hydrogen atom or a C1 to C6 linear-chain or branched-chain alkyl or alkenyl group);
D represents a hydrogen atom, a hydroxyl group, a methyl group, or a C1 to C12 linear-chain or branched-chain alkoxy or alkenyloxy group; and
E represents a hydrogen atom, a hydroxyl group, a C1 to C6 linear-chain or branched-chain alkyl or alkenyl group, or a C1 to C6 linear-chain or branched-chain alkoxy or alkenyloxy group,
wherein two or three of A¹, A², B, and E each are a hydrogen atom, with each of remaining being a group which is not a sulfo group, and in the case where D is a hydrogen atom or a methyl group, A¹ and B, or A² and B form an optionally hydroxyl group-substituted benzene ring with two adjacent carbon atoms; 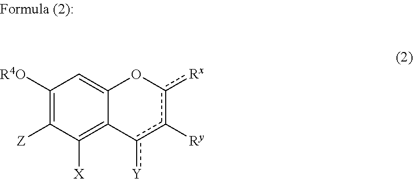
wherein,
R⁴ represents a hydrogen atom or a methyl group;
X represents a hydrogen atom, a hydroxyl group, or a methoxy group;
Y represents a hydrogen atom, an oxygen atom, a hydroxyl group, or a methoxy group;
Z represents a hydrogen atom or a C1 to C5 linear-chain or branched-chain alkyl or alkenyl group;
R^{x} represents a hydrogen atom, an oxygen atom, a hydroxy group, a methoxy group, or an aromatic hydrocarbon group, which is optionally substituted with up to three hydroxy groups or methoxy groups and which optionally forms a condensed ring with 1,3-dioxolane;
R^{y} represents a hydrogen atom, a hydroxy group, a methoxy group, or an aromatic hydrocarbon group, which is optionally substituted with up to three hydroxy groups or methoxy groups and which optionally forms a condensed ring with 1,3-dioxolane, or an arylcarbonyloxy group or aralkylcarbonyloxy group, which is optionally substituted with up to three hydroxy groups or methoxy groups;
a dotted line portion may be a double bond;
each of the combinations of the dotted line and the solid line being adjacent to R^{x} or Y denotes a double bond in the case where R^{x} or Y is an oxygen atom, and denotes a single bond in the other cases; and
Z represents a C1 to C5 linear-chain or branched-chain alkyl or alkenyl group, only when R^{x} or R^{y} is an o,p-dihydroxyaromatic hydrocarbon group, and represents a hydrogen atom in the other cases.

2. The hair treatment method according to claim 1, wherein, in step (ii), the molar ratio of component (B) to component (A) applied to hair, (B)/(A), is 0.001 or higher and lower than 2.5.

3. The hair treatment method according to claim 1, wherein step (i) includes a step of applying to the hair a hair dye composition containing one or more dyes selected from the group consisting of direct dyes and oxidative dyes.

4. The hair treatment method according to claim 3, wherein the direct dye is one or more species selected from the group consisting of an anionic dye, a nitro dye, a cationic dye, a dye having azophenol structure selected from the group consisting of HC Red 18, HC Blue 18, and HC Yellow 16, and a salt thereof.

5. The hair treatment method according to claim 3, or which method further includes a step of rinsing the hair dye composition after step (i).

6. The hair treatment method according to claim 1, which method further includes a step of allowing the hair to stand for 1 minute or more and 1 hour or less in a state in which the components (A), (B), and (C) have been applied to the hair.

7. The hair treatment method according to claim 1, wherein the heating temperature in step (iii) is 50° C. or higher and 250° C. or lower.

8. The hair treatment method according to claim 1, wherein step (ii) comprises a step of applying a hair deforming agent containing components (A), (B), and (C) to the hair.

9. The hair treatment method according to claim 8, wherein the total amount of components of the hair deforming agent which reduce hair protein, relative to the total hair deforming agent, is less than 0.1 mass %.

10. The hair treatment method according to claim 8, wherein the hair deforming agent is a one-agent type hair deforming agent.

11. The hair treatment method according to claim 10, wherein the pH of the hair deforming agent is 4 or lower.

12. The hair treatment method according to claim 8, wherein the hair deforming agent is a multi-agent type hair deforming agent including a first agent containing components (B) and (C), and a second agent containing components (A) and (C).

13. The hair treatment method according to claim 12, wherein the second agent has a pH of 4 or lower.

14. The hair treatment method according to claim 12, step (ii) is a step of applying the first agent to the hair and subsequently applying the second agent to a portion of the hair to which the first agent has been applied.

15. The hair treatment method according to claim 14, which method further includes, after application of the first agent in step (ii), a step of allowing the hair to stand for 1 minute or more and 1 hour or less.

16. The hair treatment method according to claim 14, which method further includes, after application of the second agent in step (ii), a step of allowing the hair to stand for 1 minute or more and 1 hour or less.

17. The hair treatment method according to claim 14, which method further includes, after application of the first agent in step (ii), and before application of the second agent to a portion of the hair to which the first agent has been applied, a step of rinsing the first agent applied onto the hair.

18. The hair treatment method according to claim 17, wherein the molecular weight of component (B) contained in the first agent is 140 or more and 1,000 or less.

19. The hair treatment method according to claim 1, which method includes no step of applying, to the hair, a hair deforming agent containing a reducing agent or a strongly alkaline hair treatment agent having a pH of 12 or higher and 14 or lower.

20. The hair treatment method according to claim 8, wherein the hair deforming agent has a component (A) content, relative to the total composition of the hair deforming agent and in terms of glyoxylic acid, of 1 mass % or higher and 30 mass % or lower.

21. The hair treatment method according to claim 8, wherein the hair deforming agent has a component (B) content, relative to the total composition of the hair deforming agent of 0.2 mass % or higher and 30 mass % or lower.

22. The hair treatment method according to claim 1, wherein the glyoxylamide of component (A) is N-glyoxyloylcarbocysteine or N-glyoxyloylkeratinamino acid.

23. The hair treatment method according to claim 1, wherein component (B) is component (B2)

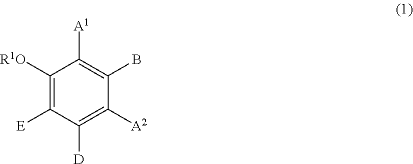

24. The hair treatment method according to claim 1, wherein component (B) is component (B3)

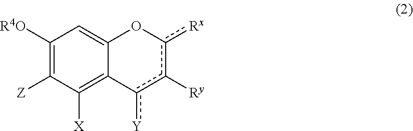

25. The hair treatment method according to claim 1, wherein component (B) is component (B1), which is resorcin.

26. The hair treatment method according to claim 1, comprising a step of heating and re-deforming the shape of hair to different shape after the step (iii).

27. The hair treatment method according to claim 12, wherein the first agent has a pH of 6 or lower.
